(19) United States
(12) Patent Application Publication
(10) Pub. No.: US 2019/0085326 A1
(43) Pub. Date: Mar. 21, 2019

(54) Negative feedback regulation of HIV-1 by gene editing strategy

(71) Applicant: Temple University - of the Commonwealth System of Higher Education, Philadelphia, PA (US)

(72) Inventors: Kamel Khalili, Bala Cynwyd, PA (US); Wenhui Hu, Cherry Hill, NJ (US); Rafal Kaminski, Philadelphia, PA (US); Thomas Malcolm, Bedminster, NJ (US)

(21) Appl. No.: 16/208,314

(22) Filed: Dec. 3, 2018

Related U.S. Application Data
(62) Division of application No. PCT/US2017/034763, filed on May 26, 2017.
(60) Provisional application No. 62/394,334, filed on Sep. 14, 2016.

(51) Int. Cl. C12N 15/11; C12N 9/22; A61P 31/18; A61K 9/00; A61K 45/06; A61K 31/7088; A61K 38/46

(52) U.S. Cl. CPC C12N 15/11; C12N 9/22; A61P 31/18; A61K 9/0014; A61K 9/0029; A61K 48/00; A61K 31/7088; A61K 38/465; C12N 2310/20; C12N 2800/80; C12N 2310/3519; A61K 45/06

(57) ABSTRACT

A CRISPR-endonuclease gene editing composition includes a guide RNA (gRNA) for targeting a specific viral sequence for cleavage by the endonuclease which introduces breaks in the double stranded DNA identified by the gRNA. Placing the gene encoding Cas9 under the control of a minimal promoter of, for example, HIV spanning the 5′-LTR, results in the activation by the HIV-1 transactivator protein, Tat. Co-expression of both a multiplex of, for example, HIV-specific gRNAs and endonuclease, e.g. Cas9, in cells results in the modification and/or excision of the segment of viral DNA, leading to the eradication of the virus in vitro and in vivo.

### CROSS-REFERENCED TO RELATED APPLICATIONS

This application is a continuation application of and claims priority to and the benefit of International Patent Application No. PCT/US17/034763 filed May 26, 2017, which claims the benefit of and priority to U.S. Provisional Patent Application 62/394,334 filed on Sep. 14, 2016 and U.S. Provisional Patent Application 62/345,549 filed on Jun. 3, 2016, the entire contents of each of which are incorporated by reference herein in its entirety.

### STATEMENT REGARDING FEDERALLY SPONSORED RESEARCH

This invention was made with government support under Grant Nos. P30MH092177 (Khalili), P01DA037830 (Khalili), R01MH092371 (Khalili), and R01NS087971 (Khalili and Hu) awarded by the National Institutes of Health. The government has certain rights in this invention.

### SEQUENCE LISTING

The instant application contains a Sequence Listing which has been submitted electronically in ASCII format and is hereby incorporated by reference in its entirety. Said ASCII copy, created on Jul. 14, 2017, is named 4941I_021_SL.txt and is 21,960 bytes in size.

### FIELD OF THE INVENTION

Embodiments of the invention are directed to gene-editing complexes in the prevention, treatment and eradication of retrovirus infections in a subject. In particular, the gene-editing complexes comprise Clustered Regularly Interspaced Short Palindromic Repeat (CRISPR)-associated endonucleases which are under the control of a minimal virus promoter that is conditionally activated by a virus transcriptional regulator.

### BACKGROUND

Soon after HIV-1 infection, the viral genome becomes integrated into the host chromosome and is rapidly expressed in CD4⁺ T-cells. HIV-1 replication leads to drastic depletion of CD4⁺ T-cells (Alimonti, J. B., et al. J. Gen. Virol. 84, 1649-1661 (2003); Okoye, A. A., Picker, L. J. Immunol Rev 254, 54-64 (2013)). Often, after the acute phase of infection, the virus enters a new phase called latency, where the integrated proviral DNA continues to be expressed and viral replication proceeds at very low levels. Under these circumstances, the weakened immune system caused by persistent viral replication progresses towards AIDS and the development of a broad range of opportunistic infections that eventually lead to death within three years if untreated (3). At the molecular level, expression of the viral genome and its replication both at the acute and chronic stages is controlled by the viral promoter that spans 450 nucleotides of the 5′-long terminal region (LTR) (Garcia, J. A., et al. EMBO J. 8, 765-778 (1989); Reddy, E. P., Dasgupta, P. Pathobiology 60, 219-224 (1992)). Cooperativity occurs between a series of cellular transcriptional factors that recognize DNA sequences within the U3 region of the 5′-LTR and the HIV-1 immediate early transcription activator, Tat, that interacts with the TAR RNA sequence positioned within the leader region of the viral transcript. These interactions are required for the robust initiation and efficient elongation of transcription from integrated copies of the viral DNA (Marcello, A., et al. IUBMB Life 51, 175-181 (2001); Roebuck, K. A. et al., Gene Expr 8, 67-84 (1999)). While current antiretroviral drugs have been effective in suppressing viral infection cycles, they have yet to contain any components that inhibit viral gene expression at the transcriptional level, supporting the notion that the integrated copies of the virus may continue to express the viral genome, albeit at very low levels, in HIV-1⁺ patients under ART (Hatano, H., et al. AIDS 24, 2535-2539 (2010); Pasternak, A. O., et al. J. Clin. Microbiol. 46, 2206-2211 (2008)). Indeed, expression of viral genes drastically elevates upon cessation of ART and allows production of viral early regulatory proteins such as Tat to orchestrate productive replication of the viral genome.

In recent years, more attention has been paid to the development of effective and safe strategies toward a cure for HIV-1/AIDS. In this respect, several approaches, including elimination of latently infected cells that serve as viral reservoirs by activation of the dormant virus and boosting immune cells, known as the shock and kill strategy. While this strategy was initially promising, it has shown limited efficacy and inconsistent outcomes (Archin, N. M., et al. J. Infect. Dis. 210, 728-735 (2014); Manson McManamy, et al. Antivir. Chem. Chemother. 23, 145-149 (2014); Siliciano, J. D. et al. J. Allergy Clin. Immunol. 134, 12-19 (2014)). More recently, the discovery of novel gene editing technologies prompted several laboratories to explore possibilities for inactivating viral DNA by introducing mutations within the various regions of the viral genome and/or cellular genes that support HIV-1 infection (Khalili, K., et al. J. Neurovirol. 21, 310-321 (2015); White, M. K., et al. Discov. Med. 19, 255-262 (2015); Yin, C., et al. AIDS 30, 1163-1170 (2016)).

### SUMMARY

Embodiments of the invention are directed to compositions for conditional activation of the CRISPR/Cas at the early stage of reactivation. These compositions completely and permanently ablate virus replication prior to productive viral replication by removing a segment of the viral gene spanning the viral promoter and/or the viral coding sequence. In embodiments, a composition comprises a nucleic acid sequence encoding a clustered regularly interspaced short palindromic repeats (CRISPR)-associated endonuclease (CRISPR/Cas) operably linked to a minimal functional viral promoter whereby the minimal viral promoter is under control of an immediate early transcriptional activator, thereby conditionally activating CRISPR/Cas at an early stage of viral replication. The isolated nucleic acid further comprises at least one guide RNA that is complementary to a target nucleic acid sequence in the virus. The CRISPR/Cas excises a segment of a viral genome, for example, the segment spanning a viral promoter and/or viral coding sequence. In these embodiments, the composition is tailored to excise any virus. In certain embodiments, the virus is a retrovirus.

Other aspects are described infra.

### BRIEF DESCRIPTION OF THE DRAWINGS

The patent or application file contains at least one drawing or photograph executed in color. Copies of this patent or patent application publication with color drawing(s) or photograph(s) will be provided by the Patent and Trademark Office upon request and payment of the necessary fee.

FIGS. 1A-1E show that the expression of Cas9 by the HIV-1 LTR promoter is stimulated by Tat leading to cleavage of the viral promoter in the presence of gRNA. FIG. 1A is a schematic representation of the full-length HIV-1 LTR and the various regulatory motifs within the enhancer and core regions, and the partial Gag gene. The extent of LTR deletion mutants that are created for expression of Cas9 is depicted. The positions of the gRNA target sequence and their distance from each other is shown. FIG. 1B shows that co-transfection of TZM-bl cells with pX260-LTR-Cas9 containing the full-length LTR (−454/+66) or its various mutants (−120/+66 or −80/+66) along with a plasmid expressing Tat (pCMV-Tat) increased the level of Tat production as tested by Western blot (top panel). Expression of housekeeping t-tubulin (middle panel) and Tat (bottom panel) are shown. FIG. 1C shows that infection of TZM-b1 cells with adenovirus expressing GFP or GFP Tat followed by transduction with lentivirus expressing Cas9 by the LTR_{-80/+66} promoter and gRNAs A/B by the U6 promoter at three different MOI of 2, 4 and 8 led to cleavage of the integrated HIV-1 LTR promoter DNA sequence and the appearance of a 205 bp DNA fragment in the TZM-b1 cells (as tested by PCR and DNA gel analysis). FIG. 1D is an SDS-PAGE illustrating the level of Cas9, β-tubulin and Tat protein expressed in TZM-bl cells as described in FIG. 1C. FIG. 1E is a graph showing results of a luciferase assay illustrating transcriptional activity of the integrated HIV-1 LTR in TZM-b1 cells after various treatments as described in FIG. 1C.

FIGS. 2A-2C show that HIV-1 infection stimulates cleavage of integrated viral DNA upon induction of Cas9. The LTR-80/+66-Cas9 reporter TZM-bl cell line transduced with three different MOI (2, 4, and 8) of lentivirus expressing gRNA A/B (LV-gRNA A/B) or control (empty LV) was infected with HIV-1_{JRFL} or HIV-1_{SF162}, and after 48 hours, cells were harvested and protein expression was determined by Western blot (FIG. 2A), the level of integrated HIV-1 LTR cleavage upon induction of Cas9 after viral infection was detected by PCR/DNA gene analysis (FIG. 2B) and transcriptional activity of the integrated HIV-1 promoter was evaluated by luciferase reporter assay (FIG. 2C).

FIGS. 3A-3C show that Tat stimulation of Cas9 cleaves integrated HIV-1 DNA in T-cells encompassing the HIV-1 reporter at a latent stage. 2D10 cells with integrated copies of LTR_{-80/+66}-Cas9 gene were transduced with control (empty LV) or LV-gRNA A/B followed by transfection with pCMV or pCMV-Tat plasmids. After 48 hours, the level of various proteins, as depicted, was determined by Western blot (FIG. 3A). The genomic DNA for assessing the state of the integrated HIV-1 DNA was determined by LTR specific PCR and the excision efficiency was determined as a percentage of ratios between truncated vs. full-length amplicon and presented in arbitrary units (AU) 0-0.5 (FIG. 3B). The level of integrated viral promoter reactivation after cleavage was assessed by flow cytometry and the representative scatter plots are shown (FIG. 3C). Red positive, propidium iodide labeled, and dead cells were excluded from the analysis.

FIGS. 4A-4C show that treatment of cells with latency reversing drugs induces Cas9 expression and cleavage of integrated viral DNA in Jurkat 2D10 cells. 2D10 cells expressing LTR-80/+66-Cas9 were treated with control (empty) or lentivirus expressing gRNAs A/B and 24 hours later cells were treated with PMA (P), TSA (T) or both (P/T) for 16 hours, as indicated. Protein studies for the expression of Cas9-Flag, α-tubulin and GFP (indicative of the integrated HIV-1 genome) was determined by Western blot (FIG. 4A). Genomic DNA for the detection of the level of excision within the integrated LTR DNA by Cas9 and gRNA A/B was assessed by PCR and the excision efficiency was determined as described in FIG. 3B legend (FIG. 4B). GFP reporter assay, by flow cytometry, and representative scatter plot is shown (FIG. 4C).

FIGS. 5A-5G show that the expression of LTR-Cas9/gRNA protects cells from new HIV-1 infection. FIGS. 5A and 5B: Jurkat cells were co-transduced with LV-gRNA A/B and Lenti-LTR_{(-80/+66)}-Cas9-Blast. The next day, cells were infected with HIV-1_{NA-3-GFP-P2A-Nef} at MOI 0.01. At days 3 and 5 of infection cells were harvested and the level of excision was assessed by LTR specific PCR using genomic DNA as a template (FIG. 5A) and quantified (FIG. 5B) as in FIGS. 3B, 3C. FIG. 5C: Direct sequencing analysis of the 205 bp DNA fragment after cloning in TA vector and selection of 10 clones designated as truncLTR 1 through 10, illustrating the positions of excision fragments compared to the control NL4-3. The positions of gRNAs corresponding to LTR A and LTR B as well as PAM sequences, and the primers used for amplification of the DNA are highlighted. FIG. 5C discloses SEQ ID NOS 42-85, respectively, in order of appearance. FIG. 5D: Agarose gels depicting results from PCR analysis for the DNA segment corresponding to UTR, Env, and control β-actin DNA in the experimental cells after 3 and 5 days of HIV-1 infection. FIG. 5E: Results from flow cytometry quantifying the percentage of positive cells (indicative of viral expression) at three different times post infection. Quantitative detection of viral DNA (FIG. 5F) and viral RNA (FIG. 5G) corresponding to the Gag sequence by TAQMAN™ in which P3-globin (for DNA) and β-actin (for RNA) were used as a reference.

FIG. 6 is a schematic representation of negative feedback regulation of HIV-1 by CRISPR/Cas9. At the early stage of viral replication, basal transcription of the viral genome results in the production of Tat protein. The association of Tat with the TAR stem loop structure within the leader of the viral transcript at the budge leads to the recruitment of several cellular regulatory proteins leading to the induction of viral transcription (solid thick arrow). At the early stage, Tat also stimulates the minimum viral promoter (depicted as ltr), which drives transcription of Cas9 gene. The newly synthesized Cas9 upon association with the various HIV-1 specific gRNAs, in turn, cleaves the viral genome and permanently inactivates LTR activity by excising a large segment of viral DNA, hence shutting down HIV-1 gene expression and replication.

FIG. 7 shows the position and nucleotide sequences of gRNA A/B targets within the LTR (highlighted in medium shade grey (green), PAM in dark gray (red)) and LTR specific primers used in PCR on TZM-bl and in vitro infected Jurkat cells genomic DNA (highlighted in light shade grey (blue)) in the reference HIV-1 NL4-3 genome (SEQ ID NO: 26). Sequences and sizes of LTR specific PCR products ((full-length (SEQ ID NO: 27) and truncated (SEQ ID NO: 30)) and predicted edited fragment (SEQ ID NO: 29).

FIG. 8 shows a representative agarose gel analyzing LTR specific PCR reactions used for quantification of Cas9/gRNA mediated LTR excision efficiency in experiments using the Jurkat 2D10 reporter cell line from FIGS. 3A-3C and 4A-4C.

FIGS. 9A-9C show the position and nucleotide composition of LTR gRNA A/B targets (highlighted in medium shade grey (green), PAM (dark grey (red)) and LTR specific primers used to analyze excision by PCR in Jurkat 2D10 cells (highlighted in light grey (blue) in the reference HIV-1 NL4-3 genome (SEQ ID NO: 26). Nucleotide sequences and sizes of amplicons ((full-length (SEQ ID NO: 31) and truncated LTR DNA (SEQ ID NO: 34)) and predicted excised DNA fragment (SEQ ID NO: 33) are shown.

FIG. 10A shows representative fluorescence microscopy images of transduced/infected Jurkat cells at day 5 of infection. Expression of BFP is indicative of the presence a vector expressing gRNAs. HIV-1 infection was monitored by the level of GFP. FIG. 10B is a graph showing a quantitative comparison of cell numbers at various time points between the control and experimental samples treated with LTR-Cas9.

FIGS. 11A-11C are graphs showing results from primary human fetal astrocytes and microglia which were transduced with lentiviral cocktails containing: lenti-LTR_{(-80/+66})-Cas9 (MOI 10), lenti-KLV-BFP-LTR A, B (MOI 3.3 of each). At day 3 post-transduction cells were infected with HIV-1_{NA-3-GFP-P2A-Nef}/VSV-G at MOI 1. One week after HIV-1 infection cells were harvested and viral expression levels were quantified by GFP expression in flow cytometry (FIG. 11A) viral DNA levels (FIG. 11B) and viral RNA (FIG. 11C) by TAQMAN qPCR and qRT-PCRs using primer set and probe specific for Gag gene.

### DETAILED DESCRIPTION

Embodiments of the invention are directed to compositions and their uses in methods for the conditional activation of the CRISPR/Cas9 at an early stage of viral reactivation by the HIV-1 transcriptional activator, Tat. These compositions permanently ablate virus replication prior to productive viral replication by removing a segment of the viral gene spanning the viral promoter and/or the viral coding sequence. Further, the use of these compositions in the methods embodied herein alleviate any concerns due to unforeseen complications that may arise by unnecessary and persistent expression of Cas9 at high levels in cells.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “anti-viral agent” as used herein, refers to any molecule that is used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof.

The term “eradication” of a retrovirus, e.g. human immunodeficiency virus (HIV), as used herein, means that that virus is unable to replicate, the genome is deleted, fragmented, degraded, genetically inactivated, or any other physical, biological, chemical or structural manifestation, that prevents the virus from being transmissible or infecting any other cell or subject resulting in the clearance of the virus in vivo. In some cases, fragments of the viral genome may be detectable, however, the virus is incapable of replication, or infection etc.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

The term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

In the context of the present invention, the following abbreviations for the commonly occurring nucleic acid bases are used, “A” refers to adenosine, “C” refers to cytosine, “G” refers to guanosine, “T” refers to thymidine, and “U” refers to uridine.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The term “polynucleotide” is a chain of nucleotides, also known as a “nucleic acid”. As used herein polynucleotides include, but are not limited to, all nucleic acid sequences which are obtained by any means available in the art, and include both naturally occurring and synthetic nucleic acids.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “percent sequence identity” or having “a sequence identity” refers to the degree of identity between any given query sequence and a subject sequence.

The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

Where any amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Compositions

Anti-retroviral therapy does not suppress low levels of viral genome expression nor does it efficiently target latently infected cells such as resting memory T cells, monocytes, macrophages, microglia, astrocytes, and gut associated lymphoid cells as described earlier. In contrast to any therapies available prior to this invention, the methods and compositions disclosed herein are useful for treatment and eradication of HIV in infected subjects at any stage of infection, or to an uninfected subject who is at risk for HIV infection.

Accordingly, the disclosed methods and compositions are useful for HIV infected subjects who are in the latent period of the infection. Moreover, when a guide RNA is associated with the CRISPR-associated endonuclease operably linked to a minimal, Tat-responsive HIV LTR promoter, as disclosed herein, the HIV genome may be excised from the host cell and eradicated. When the compositions are administered as a nucleic acid or are contained within an expression vector, the CRISPR endonuclease can be encoded by the same nucleic acid or vector as the guide RNA sequences. Alternatively, or in addition, the CRISPR endonuclease can be encoded in a physically separate nucleic acid from the guide RNA sequences or in a separate vector.

The inventors have employed the CRISPR/Cas9 technology and developed an HIV-1 specific gene editing molecule that, for the first time, excised the entire HIV-1 genome between the 5′- and 3′-LTRs from the host chromosomes of latently infected cells and protected the cells from re-infection (Khalili, K., et al. J. Neurovirol. 21, 310-321 (2015); Hu, W., et al. Proc. Natl. Acad. Sci. USA 111, 11461-11466 (2014); Kaminski, R., et al. Sci. Rep. 6: 22555 (2016)). The method of excision included use of multiplex specific guide RNAs that recognize various regions of the 5′- and 3′-LTR DNA sequences and the Cas9 endonuclease, which introduces breaks on double-stranded DNA at the sites that are complementary to the guide RNAs (Hu, W., et al. Proc. Natl. Acad. Sci. USA 111, 11461-11466 (2014); Kaminski, R., et al. Sci. Rep. 6: 22555 (2016)). After removal of viral DNA, the residual cellular DNA re-joins by cellular DNA repair (Khalili, K., et al. J. Neurovirol. 21, 310-321 (2015); White, M. K., et al. Discov. Med. 19, 255-262 (2015); Hu, W., et al. Proc. Natl. Acad. Sci. USA 111, 11461-11466 (2014); Kaminski, R., et al. Sci. Rep. 6: 22555 (2016)). The use of a multiplex of gRNAs for editing the HIV-1 genome by CRISPR technology is particularly critical in order to alleviate any concern related to the emergence of resistant virus against the initial gRNA treatment. In addition to CRISPR/Cas9 technology, more recently, recombinant based procedures have been developed with the ability to edit the HIV-1 DNA sequence from the host genome (Karpinski, J. et al. Nat. Biotechnol. 34, 401-409 (2016)).

Negative Feedback Regulation:

A viral genome, e.g. HIV integrated into an infected host cell's genome may be eliminated from such HIV infected cells utilizing an RNA-guided clustered regularly interspaced short palindromic repeat (CRISPR)-associated endonuclease such as a Cas9. Successful therapeutic gene editing using CRISPR/Cas9 enzyme and guide RNA requires efficient and specific delivery and expression of Cas9 enzyme and guide RNAs in target cells. This is difficult when the frequency of recipient cells in a tissue or population of cells is low, such as HIV infected cells in patients on highly active antiretroviral therapy (HAART).

According to the present invention, a CRISPR-associated endonuclease such as a Cas9 is placed under the control of a minimal Tat-responsive HIV LTR promoter. The endonuclease expression is thereby activated in cells containing the Tat protein. As demonstrated herein, both exogenously provided (e.g., by transfection) and endogenously produced (e.g., by reactivation of latent virus) Tat can activate (CRISPR)-associated endonuclease (e.g., Cas9) expression in cells lines when expression of the endonuclease is placed under the control of the minimal Tat-responsive HIV LTR promoter. In the studies presented further detail in the examples section, the compositions allow for the conditional activation of the CRISPR/Cas9 at the early stage of viral reactivation by the HIV-1 transcriptional activator, Tat.

This strategy completely and permanently ablates virus replication prior to productive viral replication by removing an entire viral genome or a segment of the viral gene spanning the viral promoter and/or the viral coding sequence.

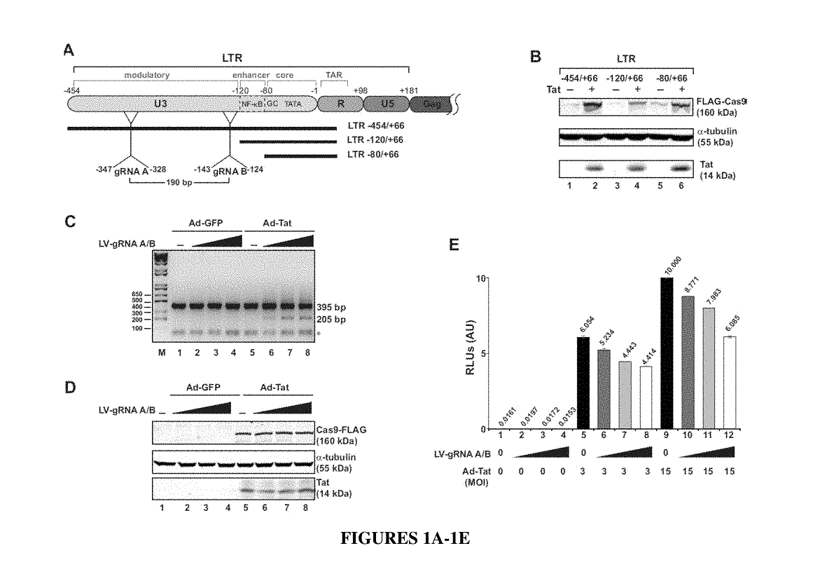

FIG. 1A is a schematic representation of the full-length HIV-1 LTR and the various regulatory motifs within the enhancer and core regions, and the partial Gag gene. The extent of LTR deletion mutants that are created for expression of Cas9 is depicted. The positions of the gRNA target sequence and their distance from each other is shown. HIV-1 LTR is approximately 640 bp in length and is divided into U3, R, and U5 regions. Transcription of the HIV-1 genome is controlled by a series of cis-acting regulatory motifs spanning the long-terminal region of the viral genome at the 5′ end. The U3 region of the viral promoter occupies −1 to −454 nucleotides, with respect to the transcription start site at +1 and has three sub-regions: modulatory, enhancer, and core. The enhancer contains the NF-κB binding site (−127 to −80). The core domain comprises the GC-rich and TATA box (−80 to +1). The R region (+1 to +98) of the LTR comprises TAR, a region for which the expressed RNA forms a stem-loop structure and provides a binding site for the viral transactivator (Krebs et al, Lentiviral LTR-directed expression, sequence variation, disease pathogenesis. Los Alamos National Laboratory HIV Sequence: Compendium, pp. 29-70.2002).

The LTRs contain all of the required signals for gene expression and are involved in the integration of a provirus into the genome of a host cell. For example, the core promoter, an enhancer, and a modulatory region are found within U3 while the TAR is found within R as shown in FIG. 1A. TAR, the binding site for Tat protein and for cellular proteins, consists of approximately the first 45 nucleotides of the viral mRNAs in HIV-1 forms a hairpin stem-loop structure. In HIV-1, the U5 region includes several sub-regions, for example, including Poly A which is involved in dimerization and genome packaging, PBS or primer binding site, Psi or the packaging signal, and DIS or dimer initiation site.

The negative feedback regulation of HIV-1 by CRISPR/Cas9 is shown in FIG. 6. At the early stage of viral replication, basal transcription of the viral genome results in the production of Tat protein. The association of Tat with the TAR stem loop structure within the leader of the viral transcript at the budge leads to the recruitment of several cellular regulatory proteins leading to the induction of viral transcription (solid thick arrow). At the early stage, Tat also stimulates the minimum viral promoter (depicted as ltr), which drives transcription of Cas9 gene. The newly synthesized Cas9 upon association with the various HIV-1 specific gRNAs, in turn, cleaves the viral genome and permanently inactivates LTR activity by excising a large segment of viral DNA, hence shutting down HIV-1 gene expression and replication.

Minimal LTR Promoter:

According to the present invention, a composition is provided comprising an isolated nucleic acid encoding a CRISPR-associated endonuclease operably linked to a minimal HIV LTR promoter comprising at least the core region and the TAR (transactivation response element) region of HIV LTR promoter. A minimal HIV LTR promoter refers to an operative functional promoter containing less than the full length HIV LTR promoter. In certain embodiments, the minimal promoter contains a TAR region. In certain embodiments, the minimal promoter comprises a core region and a TAR region. In certain embodiments, the minimal promoter comprises a core region and a TAR region without all or substantially all of the modulatory and/or enhancer regions. In another embodiment, the minimal HIV LTR promoter comprises the core region, the TAR region, and all or substantially all of the enhancer region, but does not contain any of the modulatory region. In certain embodiments, the minimal HIV LTR promoter comprises one or more mutations, modified bases, variants, locked nucleic acids combinations thereof. The minimal HIV LTR promoter is responsive to Tat protein. That is, Tat can activate the expression of the CRISPR-associated endonuclease, such as Cas9, operably linked to the minimal HIV LTR promoter. The disclosed composition may be utilized to eradicate HIV in a host cell in vitro or in vivo, inactivate HIV in a mammalian cell, treat a subject having a HIV infection, reduce the risk of HIV infection in a subject at risk for infection, and/or reduce the risk of transmission of HIV from a HIV-infected mother to her offspring. The therapeutic methods disclosed herein may be carried out in connection with other antiretroviral therapies such as HAART. The composition may be included as a part of a kit for diagnostic, research, and/or therapeutic applications.

Several advantages may be realized with the compositions containing a sequence encoding CRISPR-associated endonuclease operably linked to a minimal HIV LTR promoter containing the core region and the TAR region of HIV LTR promoter. The potential risk of toxic effects caused by the continuous expression may be alleviated and/or eliminated by limiting the expression of the CRISPR-associated endonuclease to cells with HIV gene expression and/or replication. For example, the potential to induce toxicity due to the immunogenicity of the CRISPR-associated endonuclease may be mitigated because of the low and/or intermittent expression of the endonuclease according to the present invention, while at the same time eliminate or cause self-destruction of the HIV genome in infected individuals. In addition, the present invention may provide a prophylactic strategy for at risk individuals because persistent expression of the CRISPR-associated endonuclease is minimized. Thus, the CRISPR-associated endonuclease driven by a minimal, Tat-responsive HIV LTR promoter may be utilized to provide a safe treatment of HIV infected subjects, and to vaccinate uninfected individuals who may be at risk of infection.

The minimal HIV-1 LTR promoter may comprise a nucleic acid that includes the nucleotides of positions −80 to +66 of the HIV-1 LTR promoter. In an embodiment, the minimal HIV-1 LTR promoter may comprise a nucleic acid that includes positions −120 to +66 of the HIV-1 LTR promoter. Preferably, the minimal HIV-1 LTR promoter does not contain sequences from the modulatory region. In some embodiments the promoter comprises one or more mutations, deletions, insertions, variants, derivatives or combinations thereof. The promoter may also be chimeric, comprising one or chimeric compounds.

Placing the CRISPR-associated endonuclease under control of a minimal HIV LTR promoter, as described herein, is also advantageous because a smaller-sized nucleic acid may be more readily packaged into delivery mechanisms suitable for gene therapy (e.g., retroviruses). Promoter constructs that include the modulatory region, for example, may be less suitable for gene therapy due to their size and/or variable effects on transcription of CRISPR-associated endonuclease.

As described above, the HIV genome integrates into a host genome of an individual infected with HIV. This integrated sequence is then replicated by the host. Even in the latent period, Tat may be produced by the cell. The compositions of the present invention eliminate and/or reduce the presence of the proviral polynucleotides in the host. Because the CRISPR-associated endonuclease is driven by a Tat-responsive promoter according to the present invention, any time Tat is present (e.g., produced by an infected cell), the endonuclease is produced and degrades the nascent polynucleotides. When the virus is not active, no endonuclease is produced. Thus avoided are potential toxic effects that continual expression of the endonuclease may exert on the cell and/or host.

CRISPR-Associated Endonucleases: The compositions disclosed herein may include nucleic acids encoding a CRISPR-associated endonuclease, such as Cas9. In some embodiments, one or more guide RNAs that are complementary to a target sequence of HIV may also be encoded. In bacteria, the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR RNA (crRNA). The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or Hi-promoted RNA expression vector, although cleavage efficiencies of the artificial sgRNA are lower than those for systems with the crRNA and tracrRNA expressed separately.

The CRISPR-associated endonuclease can be a Cas9 nuclease. The Cas9 nuclease can have a nucleotide sequence identical to the wild type Streptococcus pyogenes sequence. The CRISPR-associated endonuclease may be a sequence from other species, for example other Streptococcus species, such as thermophiles. The Cas9 nuclease sequence can be derived from other species including, but not limited to: Nocardiopsis dassonvillei, Streptomyces pristinaespiralis, Streptomyces viridochromogenes, Streptomyces roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficle, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Psuedomona aeruginosa, Escherichia coli, or other sequenced bacteria genomes and archaea, or other prokaryotic microorganisms may also be a source of the Cas9 sequence utilized in the embodiments disclosed herein.

The wild type Streptococcus pyogenes Cas9 sequence can be modified. The nucleic acid sequence can be codon optimized for efficient expression in mammalian cells, i.e., “humanized.” sequence can be for example, the Cas9 nuclease sequence encoded by any of the expression vectors listed in Genbank accession numbers KM099231.1 GI:669193757; KM099232.1 GI:669193761; or KM099233.1 GI:669193765. Alternatively, the Cas9 nuclease sequence can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM09923 1.1 GI:669193757; KM099232.1 GI:669193761; or KM099233.1 GI:669193765 or Cas9 amino acid sequence of PX330 or PX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and Lcyclopentyl glycine (S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

Guide RNA Sequences: The compositions and methods of the present invention may include a sequence encoding a guide RNA that is complementary to a target sequence in HIV. The genetic variability of HIV is reflected in the multiple groups and subtypes that have been described. A collection of HIV sequences is compiled in the Los Alamos HIV databases and compendiums (i.e., the sequence database web site is hitp://www.hiv.lani.gov). The methods and compositions of the invention can be applied to HIV from any of those various groups, subtypes, and circulating recombinant forms. These include for example, the HIV-1 major group (often referred to as Group M) and the minor groups, Groups N, O, and P, as well as but not limited to, any of the following subtypes, A, B, C, D, F, G, H, J and K. or group (for example, but not limited to any of the following Groups, N, O and P) of HIV.

The guide RNA can be a sequence complimentary to a coding or a non-coding sequence (i.e., a target sequence). For example, the guide RNA can be a sequence that is complementary to a HIV long terminal repeat (LTR) region other than the portions that are utilized informing the minimal Tat-responsive promoter that is operably linked to the Cas9 gene. The guide RNA cannot target the sequence corresponding to the minimal Tat-responding HIV-1 LTR promoter as disclosed herein because it would result in degradation of the construct itself, thereby potentially removing the advantages rendered by the CRISPR-associated endonuclease driven by the minimal HIV LTR promoter. Thus, a guide RNA can include a sequence found within an HIV-1 U3, R, and/or U5 region reference sequence or consensus sequence, without selecting a sequence that is a part of the minimal Tat-responsive HIV promoter.

In some embodiments, the guide RNA can be a sequence complementary to a coding sequence such as a sequence encoding one or more viral structural proteins (e.g., gag, pol, env, and tat). Thus, the sequence can be complementary to sequence within the gag polyprotein, e.g., MA (matrix protein, p17); CA (capsid protein, p24); NC (nucleocapsid protein, p7); and P6 protein; pol, e.g., reverse transcriptase (RT) and RNase H, integrase (IN), and HIV protease (PR); env, e.g., gp160, or a cleavage product of gp160, e.g., gp120 or SU, and gp41 or TM; or tat, e.g., the 72-amino acid one-exon Tat or the 86-101 amino-acid two-exon Tat. In some embodiments, the guide RNA can be a sequence complementary to a sequence encoding an accessory protein, including for example, vif, n willef (negative factor) vpu (Virus protein U) and tev.

In some embodiments, the guide RNA sequence can be a sequence complementary to a structural or regulatory element (i.e., a target sequence) such as RRE, PE, SLIP, CRS (Cis-acting repressive sequences), and/or INS. RRE (Rev responsive element) is an RNA element encoded within the env region of HIV and includes approximately 200 nucleotides (positions 7710 to 8061 from the start of transcription in HIV-1, spanning the border of gp120 and gp41). PE (Psi element) corresponds to a set of 4 stem-loop structures preceding and overlapping the Gag start codon. SLIP is a TTTTTT “slippery site” followed by a stem-loop structure. CRS (Cis-acting repressive sequences). INS (Inhibitory/Instability RNA sequences) may be found for example, at nucleotides 414 to 631 in the gag region of HIV-1.

The guide RNA sequence can be a sense or anti-sense sequence. The guide RNA sequence generally includes a PAM. The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. For example, Cas9 from S. thermophilus requires 5′-NNAGAA for CRISPR 1 and 5′-NGGNG for CRISPR3) and Neiseria menigiditis requires 5′-NNNNGATT). The specific sequence of the guide RNA may vary, but, regardless of the sequence, useful guide RNA sequences will be those that minimize off-target effects while achieving high efficiency and complete ablation of the genomically integrated HIV provirus. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, 8, about 39, about 40, about 45, about 50, about 55, about 60 or more nucleotides. Useful selection methods identify regions having extremely low homology between the foreign viral genome and host cellular genome including endogenous retroviral DNA, include bioinformatic screening using 12-bp+NGG target-selection criteria to exclude off-target human transcriptome or (even rarely) untranslated-genomic sites; avoiding transcription factor binding sites within the HIV-1 LTR promoter (potentially conserved in the host genome); selection of LTR-A- and -B-directed, 30-bp guide RNAs and also pre-crRNA system reflecting the original bacterial immune mechanism to enhance specificity/efficiency versus 20-bp guide RNA-, chimeric crRNA-tracRNA-based system and WGS, Sanger sequencing and SURVEYOR assay, to identify and exclude potential off-target effects.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs, for example a combination of sequences in U3, R, or U5, without selecting a sequence that is a part of the minimal Tat-responsive HIV promoter. When the compositions are administered in an expression vector, the guide RNAs can be encoded by a single vector. Alternatively, multiple vectors can be engineered to each include two or more different guide RNAs. Useful configurations will result in the excision of viral sequences between cleavage sites resulting in the ablation of HIV genome or HIV protein expression. Thus, the use of two or more different guide RNAs promotes excision of the viral sequences between the cleavage sites recognized by the CRISPR endonuclease. The excised region can vary in size from a single nucleotide to several thousand nucleotides.

Modified or Mutated Nucleic Acid Sequences: In some embodiments, any of the nucleic acid sequences may be modified or derived from a native nucleic acid sequence, for example, by introduction of mutations, deletions, substitutions, modification of nucleobases, backbones and the like. The nucleic acid sequences include the vectors, gene-editing agents, gRNAs, etc. Examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH, —N(CH₃)—O—CH₂ [known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,). The amide backbones disclosed by De Mesmaeker et al. Acc. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

The nucleic acid sequences may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N⁶ (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278).

Another modification of the nucleic acid sequences of the invention involves chemically linking to the nucleic acid sequences one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶Nmethyladenosine; N⁶, N⁶-dimethyladenosine; N⁶,2′-O-trimethyladenosine; 2 methylthio-N⁶Nisopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶ threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1,2′-O-dimethyl inosine; 2′-O-methyl guanosine; 1-methyl guanosine; N²-methyl guanosine; N², N²-dimethyl guanosine; N², 2′-O-dimethyl guanosine; N², N², 2′-O-trimethyl guanosine; 2′-O-ribosyl guanosine (phosphate); 7-methyl guanosine; N², 7-dimethyl guanosine; N², N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

Isolated nucleic acid molecules can be produced by standard techniques. For example, PCR techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring portion of a Cas9-encoding DNA.

Two nucleic acids or the polypeptides they encode may be described as having a certain degree of identity to one another. For example, a Cas9 protein and a biologically active variant thereof may be described as exhibiting a certain degree of identity. Alignments may be assembled by locating short Cas9 sequences in the Protein Information Research (PIR) site (pir.georgetown.edu), followed by analysis with the “short nearly identical sequences” Basic Local Alignment Search Tool (BLAST) algorithm on the NCBI website (ncbi.nlm.nih.gov/blast).

A percent sequence identity to Cas9 can be determined and the identified variants may be utilized as a CRISPR-associated endonuclease and/or assayed for their efficacy as a pharmaceutical composition. A naturally occurring Cas9 can be the query sequence and a fragment of a Cas9 protein can be the subject sequence. Similarly, a fragment of a Cas9 protein can be the query sequence and a biologically active variant thereof can be the subject sequence. To determine sequence identity, a query nucleic acid or amino acid sequence can be aligned to one or more subject nucleic acid or amino acid sequences, respectively, using the computer program ClustalW (version 1.83, default parameters), which allows alignments of nucleic acid or protein sequences to be carried out across their entire length (global alignment). See Chenna et al., Nucleic Acids Res. 31:3497-3500, 2003.

Recombinant Constructs and Delivery Vehicles: Recombinant constructs are also provided herein and can be used to transform cells in order to express Cas9 under the control of a minimal Tat-responsive HIV LTR promoter. Recombinant constructs may similarly be utilized to express a guide RNA complementary to a target sequence in HIV. A recombinant nucleic acid construct comprises a nucleic acid encoding a Cas9 and/or a guide RNA complementary to a target sequence in HIV as described herein, operably linked to a regulatory region suitable for expressing the Cas9 and/or a guide RNA complementary to a target sequence in HIV in the cell. It will be appreciated that a number of nucleic acids can encode a polypeptide having a particular amino acid sequence. The degeneracy of the genetic code is well known in the art. For many amino acids, there is more than one nucleotide triplet that serves as the codon for the amino acid. For example, codons in the coding sequence for Cas9 can be modified such that optimal expression in a particular organism is obtained, using appropriate codon bias tables for that organism.

Nucleic acids as described herein may be contained in vectors. Vectors can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FLAG™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col El, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Several delivery methods may be utilized in conjunction with the minimal Tat-responsive HIV LTR promoter operably linked to the Cas9 gene for in vitro (cell cultures) and in vivo (animals and patients) systems. In one embodiment, a lentiviral gene delivery system may be utilized. Such a system offers stable, long term presence of the gene in dividing and non-dividing cells with broad tropism and the capacity for large DNA inserts. (Dull et al, J Virol, 72:8463-8471 1998). In an embodiment, adeno-associated virus (AAV) may be utilized as a delivery method. AAV is a non-pathogenic, single-stranded DNA virus that has been actively employed in recent years for delivering therapeutic gene in in vitro and in vivo systems (Choi et al, Curr Gene Ther, 5:299-310, 2005). An example non-viral delivery method may utilize nanoparticle technology. This platform has demonstrated utility as a pharmaceutical in vivo. Nanotechnology has improved transcytosis of drugs across tight epithelial and endothelial barriers. It offers targeted delivery of its payload to cells and tissues in a specific manner (Allen and Cullis, Science, 303:1818-1822, 1998).

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses Ad, AAV, lentivirus, and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors is known in the art and are generally available. A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Additional vectors include viral vectors, fusion proteins and chemical conjugates. Retroviral vectors include Moloney murine leukemia viruses and HIV-based viruses. One HIV based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

The polynucleotides disclosed herein may be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another delivery method is to use single stranded DNA producing vectors which can produce the expressed products intracellularly. See for example, Chen et al, BioTechniques, 34: 167-171 (2003), which is incorporated herein, by reference, in its entirety.

Another delivery method is to use single stranded DNA producing vectors which can produce the expressed products intracellularly. See for example, Chen et al, BioTechniques, 34: 167-171 (2003), which is incorporated herein, by reference, in its entirety. The polynucleotides disclosed herein may be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989).

In certain embodiments of the invention, non-viral vectors may be used to effectuate transfection. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those described in U.S. Pat. No. 7,166,298 to Jessee or U.S. Pat. No. 6,890,554 to Jesse, the contents of each of which are incorporated by reference. Delivery can be to cells (e.g. in vitro or ex vivo administration) or target tissues (e.g. in vivo administration).

Synthetic vectors are typically based on cationic lipids or polymers which can complex with negatively charged nucleic acids to form particles with a diameter in the order of 100 nm. The complex protects nucleic acid from degradation by nuclease. Moreover, cellular and local delivery strategies have to deal with the need for internalization, release, and distribution in the proper subcellular compartment. Systemic delivery strategies encounter additional hurdles, for example, strong interaction of cationic delivery vehicles with blood components, uptake by the reticuloendothelial system, kidney filtration, toxicity and targeting ability of the carriers to the cells of interest. Modifying the surfaces of the cationic non-virals can minimize their interaction with blood components, reduce reticuloendothelial system uptake, decrease their toxicity and increase their binding affinity with the target cells. Binding of plasma proteins (also termed opsonization) is the primary mechanism for RES to recognize the circulating nanoparticles. For example, macrophages, such as the Kupffer cells in the liver, recognize the opsonized nanoparticles via the scavenger receptor.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are commonly latently infected reservoirs of HIV infections. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprising a sequence encoding CRISPR/Cas and/or a guide RNA complementary to a target sequence of HIV, as described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol modified (PEGylated) low molecular weight LPEI. In some embodiments, the compositions can be formulated as a nanoparticle encapsulating the compositions embodied herein. L-PEI has been used to efficiently deliver genes in vivo into a wide range of organs such as lung, brain, pancreas, retina, bladder as well as tumor. L-PEI is able to efficiently condense, stabilize and deliver nucleic acids in vitro and in vivo.

In some embodiments, delivery of vectors can also be mediated by exosomes. Exosomes are lipid nanovesicles released by many cell types. They mediate intercellular communication by transporting nucleic acids and proteins between cells. Exosomes contain RNAs, miRNAs, and proteins derived from the endocytic pathway. They may be taken up by target cells by endocytosis, fusion, or both. Exosomes can be harnessed to deliver nucleic acids to specific target cells.

The expression constructs of the present invention can also be delivered by means of nanoclews. Nanoclews are a cocoon-like DNA nanocomposites (Sun, et al., J. Am. Chem. Soc. 2014, 136:14722-14725). They can be loaded with nucleic acids for uptake by target cells and release in target cell cytoplasm. Methods for constructing nanoclews, loading them, and designing release molecules can be found in Sun, et al. (Sun W, et al., J. Am. Chem. Soc. 2014, 136:14722-14725; Sun W, et al., Angew. Chem. Int. Ed. 2015: 12029-12033.)

The nucleic acids and vectors may also be applied to a surface of a device (e.g., a catheter) or contained within a pump, patch, or any other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments of the invention, liposomes are used to effectuate transfection into a cell or tissue. The pharmacology of a liposomal formulation of nucleic acid is largely determined by the extent to which the nucleic acid is encapsulated inside the liposome bilayer. Encapsulated nucleic acid is protected from nuclease degradation, while those merely associated with the surface of the liposome is not protected. Encapsulated nucleic acid shares the extended circulation lifetime and biodistribution of the intact liposome, while those that are surface associated adopt the pharmacology of naked nucleic acid once they disassociate from the liposome. Nucleic acids may be entrapped within liposomes with conventional passive loading technologies, such as ethanol drop method (as in SALP), reverse-phase evaporation method, and ethanol dilution method (as in SNALP).

Liposomal delivery systems provide stable formulation, provide improved pharmacokinetics, and a degree of ‘passive’ or ‘physiological’ targeting to tissues. Encapsulation of hydrophilic and hydrophobic materials, such as potential chemotherapy agents, are known. See for example U.S. Pat. No. 5,466,468 to Schneider, which discloses parenterally administrable liposome formulation comprising synthetic lipids; U.S. Pat. No. 5,580,571, to Hostetler et al. which discloses nucleoside analogues conjugated to phospholipids; U.S. Pat. No. 5,626,869 to Nyqvist, which discloses pharmaceutical compositions wherein the pharmaceutically active compound is heparin or a fragment thereof contained in a defined lipid system comprising at least one amphiphatic and polar lipid component and at least one nonpolar lipid component.

Liposomes and polymerosomes can contain a plurality of solutions and compounds. In certain embodiments, the complexes of the invention are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, Langmuir 21(20):9183-6.

In some embodiments of the invention, non-viral vectors are modified to effectuate targeted delivery and transfection. PEGylation (i.e. modifying the surface with polyethyleneglycol) is the predominant method used to reduce the opsonization and aggregation of non-viral vectors and minimize the clearance by reticuloendothelial system, leading to a prolonged circulation lifetime after intravenous (i.v.) administration. PEGylated nanoparticles are therefore often referred as “stealth” nanoparticles. The nanoparticles that are not rapidly cleared from the circulation will have a chance to encounter infected cells.

In some embodiments of the invention, targeted controlled-release systems responding to the unique environments of tissues and external stimuli are utilized. Gold nanorods have strong absorption bands in the near-infrared region, and the absorbed light energy is then converted into heat by gold nanorods, the so-called “photothermal effect”. Because the near-infrared light can penetrate deeply into tissues, the surface of gold nanorod could be modified with nucleic acids for controlled release. When the modified gold nanorods are irradiated by near-infrared light, nucleic acids are released due to thermo-denaturation induced by the photothermal effect. The amount of nucleic acids released is dependent upon the power and exposure time of light irradiation.

Regardless of whether compositions are administered as nucleic acids or polypeptides, they are formulated in such a way as to promote uptake by the mammalian cell. Useful vector systems and formulations are described above. In some embodiments the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems.

In other embodiments, the compositions comprise a cell which has been transformed or transfected with one or more CRISPR/Cas vectors and gRNAs. In some embodiments, the methods of the invention can be applied ex vivo. That is, a subject's cells can be removed from the body and treated with the compositions in culture to excise, for example, HIV sequences and the treated cells returned to the subject's body. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

Transduced cells are prepared for reinfusion according to established methods. After a period of about 2-4 weeks in culture, the cells may number between 1×10⁶ and 1×10¹⁰. In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent. For administration, cells of the present invention can be administered at a rate determined by the LD₅₀ of the cell type, and the side effects of the cell type at various concentrations, as applied to the mass and overall health of the patient. Administration can be accomplished via single or divided doses. Adult stem cells may also be mobilized using exogenously administered factors that stimulate their production and egress from tissues or spaces that may include, but are not restricted to, bone marrow or adipose tissues.

Pharmaceutical Compositions

As described above, the compositions of the present invention can be prepared in a variety of ways known to one of ordinary skill in the art. Regardless of their original source or the manner in which they are obtained, the compositions disclosed herein can be formulated in accordance with their use. For example, the nucleic acids and vectors described above can be formulated within compositions for application to cells in tissue culture or for administration to a patient or subject. Any of the pharmaceutical compositions of the invention can be formulated for use in the preparation of a medicament, and particular uses are indicated below in the context of treatment, e.g., the treatment of a subject having an HIV infection or at risk for contracting and HIV infection. When employed as pharmaceuticals, any of the nucleic acids and vectors can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

The pharmaceutical compositions may contain, as the active ingredient, nucleic acids and vectors described herein in combination with one or more pharmaceutically acceptable carriers. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are commonly latently infected reservoirs of HIV infection, for example, brain macrophages, microglia, astrocytes, and gut-associated lymphoid cells. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprising a sequence encoding a CRISPR-associated endonuclease and optionally a guide RNA is operably linked to the minimal Tat-responsive HIV LTR promoter as described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycolmodified (PEGylated) low molecular weight LPEI.

The nucleic acids and vectors may also be applied to a surface of a device (e.g., a catheter) or contained within a pump, patch, or other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions may be formulated as a topical gel for blocking sexual transmission of HIV. The topical gel can be applied directly to the skin or mucous membranes of the male or female genital region prior to sexual activity. Alternatively, or in addition the topical gel can be applied to the surface or contained within a male or female condom or diaphragm.

In some embodiments, the compositions can be formulated as a nanoparticle encapsulating a nucleic acid encoding Cas9 or a variant Cas9 operably linked to a minimal HIV LTR promoter. The nucleic acid may additionally encode a guide RNA sequence complementary to a target HIV.

The present formulations can encompass a vector encoding Cas9 and a guide RNA sequence complementary to a target HIV. The guide RNA sequence can include a sequence complementary to a single target region or it can include any combination of sequences complementary to multiple target regions as described earlier. Alternatively, the sequence encoding Cas9 driven by the minimal HIV LTR promoter and the sequence encoding the guide RNA sequence can be on separate vectors.

The compositions disclosed herein are generally and variously useful for treatment of a subject having an HIV infection. The methods are useful for targeting any HIV, for example, HIV-1 and HIV-2, and also SIV, and any circulating recombinant form thereof. A subject is effectively treated whenever a clinically beneficial result ensues. This may mean, for example, a complete resolution of the symptoms of a disease, a decrease in the severity of the symptoms of the disease, or a slowing of the disease's progression. These methods can further include the steps of a) identifying a subject (e.g., a patient and, more specifically, a human patient) who has an HIV infection; and b) providing to the subject a composition comprising a nucleic acid encoding a CRISPR-associated nuclease, e.g. Cas9, under control of the minimal Tat-responsive HIV LTR promoter. The methods may further include providing to the subject a sequence encoding a guide RNA complementary to an HIV target sequence, e.g. an HIV LTR.

A subject can be identified using standard clinical tests, for example, immunoassays to detect the presence of HIV antibodies or the HIV polypeptide p24 in the subject's serum, or through HIV nucleic acid amplification assays. An amount of such a composition provided to the subject that results in a complete resolution of the symptoms of the infection, a decrease in the severity of the symptoms of the infection, or a slowing of the infection's progression is considered a therapeutically effective amount. The present methods may also include a monitoring step to help optimize dosing and scheduling as well as predict outcome. In some methods of the present invention, one can first determine whether a patient has a latent HIV infection, and then make a determination as to whether or not to treat the patient with one or more of the compositions described herein. Monitoring can also be used to detect the onset of drug resistance and to rapidly distinguish responsive patients from nonresponsive patients. In some embodiments, the methods can further include the step of determining the nucleic acid sequence of the particular HIV harbored by the patient and then designing the guide RNA to be complementary to those particular sequences. For example, one can determine the nucleic acid sequence of a subject's LTR U3, R, or U5 region and then design one or more guide RNAs to be precisely complementary to the patient's sequences, again without selecting a sequence that is a part of the minimal Tat-responsive HIV promoter.

The compositions are also useful for the treatment, for example, as a prophylactic treatment, of a subject at risk for having a retroviral infection, e.g., an HIV infection. These methods can further include the steps of a) identifying a subject at risk for having an HIV infection; b) providing to the subject a composition comprising a nucleic acid encoding a CRISPR-associated nuclease, e.g., Cas9, under control of a minimal Tat-responsive HIV-1 LTR promoter. The sequence may additionally encode for a guide RNA complementary to an HIV target sequence, e.g. an HIV LTR. A subject at risk for having an HIV infection can be, for example, any sexually active individual engaging in unprotected sex, i.e., engaging in sexual activity without the use of a condom; a sexually active individual having another sexually transmitted infection; an intravenous drug user; or an uncircumcised man. A subject at risk for having an HIV infection can be, for example, an individual whose occupation may bring him or her into contact with HIV-infected populations, e.g., healthcare workers or first responders. A subject at risk for having an HIV infection can be, for example, an inmate in a correctional setting or a sex worker, that is, an individual who uses sexual activity for income employment or nonmonetary items such as food, drugs, or shelter.

The compositions can also be administered to a pregnant or lactating woman having an HIV infection in order to reduce the likelihood of transmission of HIV from the mother to her offspring. A pregnant woman infected with HIV can pass the virus to her offspring transplacentally in utero, at the time of delivery through the birth canal or following delivery, through breast milk. The compositions disclosed herein can be administered to the HIV infected mother either prenatally, perinatally or postnatally during the breast-feeding period, or any combination of prenatal, perinatal, and postnatal administration. Compositions can be administered to the mother along with standard antiretroviral therapies as described below. In some embodiments, the compositions of the invention are also administered to the infant immediately following delivery and, in some embodiments, at intervals thereafter. The infant also can receive standard antiretroviral therapy.

The compositions may be administered to an individual who is not infected with HIV to prevent infection with HIV. The composition may include delivering a therapeutically effective amount of the pharmaceutical composition. The pharmaceutical composition may include a sequence encoding a CRISPR-associated endonuclease and at least the core region of a HIV LTR promoter and a TAR region of the minimal Tat-responsive HIV LTR promoter as described above.

The methods disclosed herein can be applied to a wide range of species, e.g., humans, non-human primates (e.g., monkeys), horses or other livestock, dogs, cats, ferrets or other mammals kept as pets, rats, mice, or other laboratory animals.

The methods of the invention can be expressed in terms of the preparation of a medicament. Accordingly, the invention encompasses the use of the agents and compositions described herein in the preparation of a medicament. The compounds described herein are useful in therapeutic compositions and regimens or for the manufacture of a medicament for use in treatment of diseases or conditions as described herein.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

An effective amount of any composition provided herein can be administered to an individual in need of treatment. An effective amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Any method known to those in the art can be used to determine if a particular response is induced. Clinical methods that can assess the degree of a particular disease state can be used to determine if a response is induced. The particular methods used to evaluate a response will depend upon the nature of the patient's disorder, the patient's age, and sex, other drugs being administered, and the judgment of the attending clinician.

The compositions may also be administered with another therapeutic agent, for example, an anti-retroviral agent, used in HAART, chemotherapeutic agents, activators of HIV transcription, e.g. PMA, TSA, and the like. Antiretroviral agents may include reverse transcriptase inhibitors (e.g., nucleoside/nucleotide reverse transcriptase inhibitors, zidovudine, emtricitibine, lamivudine and tenoifvir; and non-nucleoside reverse transcriptase inhibitors such as efavarenz, nevirapine, rilpivirine); protease inhibitors, e.g., tipiravir, darunavir, indinavir; entry inhibitors, e.g., maraviroc; fusion inhibitors, e.g., enfuviritide; or integrase inhibitors e.g., raltegrivir, dolutegravir. Antiretroviral agents may also include multi-class combination agents for example, combinations of emtricitabine, efavarenz, and tenofivir; combinations of emtricitabine; rilpivirine, and tenofivir; or combinations of elvitegravir, cobicistat, emtricitabine and tenofivir.

In addition, one or more agents which alleviate any other symptoms that may be associated with the virus infection, e.g. fever, chills, headaches, secondary infections, can be administered in concert with, or as part of the pharmaceutical composition or at separate times. These agents comprise, without limitation, an anti-pyretic agent, anti-inflammatory agent, chemotherapeutic agent, or combinations thereof.

In certain embodiments, the anti-viral agent comprises therapeutically effective amounts of: antibodies, aptamers, adjuvants, anti-sense oligonucleotides, chemokines, cytokines, immune stimulating agents, immune modulating molecules, B-cell modulators, T-cell modulators, NK cell modulators, antigen presenting cell modulators, enzymes, siRNA's, interferon, ribavirin, protease inhibitors, anti-sense oligonucleotides, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, vaccines or combinations thereof.

The immune-modulating molecules comprise, but are not limited to cytokines, lymphokines, T cell co-stimulatory ligands, etc. An immune-modulating molecule positively and/or negatively influences the humoral and/or cellular immune system, particularly its cellular and/or non-cellular components, its functions, and/or its interactions with other physiological systems. The immune-modulating molecule may be selected from the group comprising cytokines, chemokines, macrophage migration inhibitory factor (MIF; as described, inter alia, in Bernhagen (1998), Mol Med 76(3-4); 151-61 or Metz (1997), Adv Immunol 66, 197-223), T-cell receptors or soluble MHC molecules. Such immune-modulating effector molecules are well known in the art and are described, inter alia, in Paul, “Fundamental immunology”, Raven Press, New York (1989). In particular, known cytokines and chemokines are described in Meager, “The Molecular Biology of Cytokines” (1998), John Wiley & Sons, Ltd., Chichester, West Sussex, England; (Bacon (1998). Cytokine Growth Factor Rev 9(2):167-73; Oppenheim (1997). Clin Cancer Res 12, 2682-6; Taub, (1994) Ther. Immunol. 1(4), 229-46 or Michiel, (1992). Semin Cancer Biol 3(1), 3-15).

Immune cell activity that may be measured include, but is not limited to, (1) cell proliferation by measuring the DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

Also of interest are enzymes present in the lytic package that cytotoxic T lymphocytes or LAK cells deliver to their targets. Perforin, a pore-forming protein, and Fas ligand are major cytolytic molecules in these cells (Brandau et al., Clin. Cancer Res. 6:3729, 2000; Cruz et al., Br. J. Cancer 81:881, 1999). CTLs also express a family of at least 11 serine proteases termed granzymes, which have four primary substrate specificities (Kam et al., Biochim. Biophys. Acta 1477:307, 2000). Low concentrations of streptolysin O and pneumolysin facilitate granzyme B-dependent apoptosis (Browne et al., Mol. Cell Biol. 19:8604, 1999).

Other suitable effectors encode polypeptides having activity that is not itself toxic to a cell, but renders the cell sensitive to an otherwise nontoxic compound—either by metabolically altering the cell, or by changing a non-toxic prodrug into a lethal drug. Exemplary is thymidine kinase (tk), such as may be derived from a herpes simplex virus, and catalytically equivalent variants. The HSV tk converts the anti-herpetic agent ganciclovir (GCV) to a toxic product that interferes with DNA replication in proliferating cells.

Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks. The therapeutic agents may be administered under a metronomic regimen, e.g., continuous low-doses of a therapeutic agent.

Dosage, toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As described, a therapeutically effective amount of a composition (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

The compositions described herein are suitable for use in a variety of drug delivery systems described above. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue specific antibody. The liposomes will be targeted to and taken up selectively by the organ.

Also provided, are methods of inactivating a retrovirus, for example a lentivirus such as a human immunodeficiency virus, a simian immunodeficiency virus, a feline immunodeficiency virus, or a bovine immunodeficiency virus in a mammalian cell. The human immunodeficiency virus can be HIV-1 or HIV-2. The human immunodeficiency virus can be a chromosomally integrated provirus. The mammalian cell can be any cell type infected by HIV, including, but not limited to CD4⁺ lymphocytes, macrophages, fibroblasts, monocytes, T lymphocytes, B lymphocytes, natural killer cells, dendritic cells such as Langerhans cells and follicular dendritic cells, hematopoietic stem cells, endothelial cells, brain microglial cells, astrocytes and gastrointestinal epithelial cells. Such cell types include those cell types that are typically infected during a primary infection, for example, a CD4⁺ lymphocyte, a macrophage, a monocyte or a Langerhans cell, as well as those cell types that make up latent HIV reservoirs, i.e., a latently infected cell.

The methods can include exposing and/or contacting the cell to a composition comprising an isolated nucleic acid encoding a CRISPR-associated endonuclease operably linked to a minimal HIV LTR promoter containing the core region and the TAR region of the HIV LTR promoter. The isolated nucleic acid may further encode one or more guide RNAs wherein the guide RNA is complementary to a target nucleic acid sequence in the retrovirus. The contacting step can take place in vivo, that is, the compositions can be administered directly to a subject having HIV infection. The methods are not so limited however, and the contacting step can take place ex vivo. For example, a cell or plurality of cells, or a tissue explant, can be removed from a subject having an HIV infection and placed in culture, and then contacted with a composition comprising a CRISPR-associated endonuclease operably linked to a minimal HIV LTR promoter and optionally a guide RNA wherein the guide RNA is complementary to a nucleic acid sequence in HIV. As described above, a pharmaceutical composition may include a nucleic acid encoding a CRISPR-associated endonuclease operably linked to a minimal Tat-responsive HIV LTR promoter.

The compositions are formulated in such a way as to promote uptake by the mammalian cell. Useful vector systems and formulations are described above. In some embodiments the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems.

Standard methods, for example, immunoassays to detect the CRISPR-associated endonuclease, or nucleic acid-based assays such as PCR to detect the guide RNA, can be used to confirm cell has taken up and/or expressed the protein into which it has been introduced. The engineered cells can then be reintroduced into the subject from whom they were derived as described below.

In other embodiments, the compositions comprise a cell which has been transformed or transfected with one or more Cas9/minimal Tat-responsive HIV LTR promoter vectors. In some embodiments, the methods of the invention can be applied ex vivo. That is, a subject's cells can be removed from the body and treated with the compositions in culture to excise HIV sequences and the treated cells returned to the subject's body. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

The compositions described herein can be packaged in suitable containers labeled, for example, for use as a therapy to treat a subject having a retroviral infection, for example, an HIV infection or a subject at for contracting a retroviral infection, for example, an HIV infection. The containers can include a composition comprising a nucleic acid sequence encoding a CRISPR-associated endonuclease, for example, a Cas9 endonuclease, and a minimal Tat-responsive HIV LTR promoter as described earlier. The sequence may additionally encode a guide RNA complementary to a target sequence in a HIV, or a vector encoding that nucleic acid, and one or more of a suitable stabilizer, carrier molecule, flavoring, and/or the like, as appropriate for the intended use. Accordingly, packaged products (e.g., sterile containers containing one or more of the compositions described herein and packaged for storage, shipment, or sale at concentrated or ready-to-use concentrations) and kits, including at least one of the disclosed compositions. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which prophylaxis or treatment is required. In some embodiments, the kits can include one or more additional antiretroviral agents, for example, a reverse transcriptase inhibitor, a protease inhibitor or an entry inhibitor. The additional agents can be packaged together in the same container as a nucleic acid sequence encoding a CRISPR-associated endonuclease, for example, a Cas9 endonuclease, operably linked to a minimal HIV LTR promoter and optionally a guide RNA complementary to a target sequence in a HIV, or a vector encoding that nucleic acid or they can be packaged separately.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions can be provided in a concentrated form with a diluent and instructions for dilution.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, applicants do not admit any particular reference is “prior art” to their invention.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

### Example 1: Negative Feedback Regulation of HIV-1 by Gene Editing

In the studies presented here, the gene editing technique was refined and a new strategy was developed that allows conditional activation of the CRISPR/Cas9 at an early stage of viral reactivation by the HIV-1 transcriptional activator, Tat. This new strategy permanently ablates virus replication prior to productive viral replication by removing a segment of the viral gene spanning the viral promoter and/or the viral coding sequence. Further, this strategy alleviates any concerns due to unforeseen complications that may arise by unnecessary and persistent expression of Cas9 at high levels in cells.

Materials and Methods

Plasmid Preparation.

Full length and truncated LTR promoter sequences were obtained by PCR using pNL4-3 HIV vector (NIH AIDS Reagent Program #114) as a template and the primers are listed in Table 1 (SEQ ID NOS: 1-4). PCR products were gel purified and directly subcloned in TA vector (Invitrogen), then excised with Kpnl or Xba1 and Nco1 restriction enzymes and ligated into Kpn1-Nco1 or Xba1-Nco1 digested pX260-U6-DR-BB-DR-Cbh-NLS-hSpCas9-NLS-H1-shorttracr-PGK-puro plasmid (Addgene #42229). As a result, the original Cbh promoter (Xba1-Kpn1-Cbh-Nco1) in pX260 plasmid was removed and replaced with one of the LTR promoters (Xba1- or Kpn1-LTR-Nco1). To create a lentiviral LTR-Cas9 construct, lentiCas9-Blast (Addgene #52962) was treated with Nhe1/Xba1, as a result, EFS promoter sequence was removed and replaced by Nhe1-LTR-80/+66-Xbal digested PCR product creating Lenti-LTR-80/+66-Cas9-Blast plasmid (Table 1; SEQ ID NOS: 5, 6). pKLV-U6-LTR A/B-PGKpuro2ABFP lentiviral plasmid was described previously (Kaminski, R., et al. Sci. Rep. 6: 22555 (2016)). pcDNA3.1 control vector purchased from Invitrogen, and pCMV-Tat86 has been described previously (Gallia, G. L., et al. Proc. Natl. Acad. Sci. USA 96, 11572-11577 (1999)). pKLV-U6-LTR A/B-PGKpuro2ABFP were packaged into lentiviral particles by co-transfection of HEK293T cells with pMDLg/pRRE (Addgene 12251), pRSV-Rev (Addgene 12253) and pCMV-VSV-G (Addgene 8454). For packaging Cas9 into lentiviral particles following vectors were used: Lenti-LTR-80/+66-Cas9-Blast, psPAX2 (Addgene 12260) and pCMV-VSV-G (Addgene 8454).

Cell Culture. The TZM-bl reporter cell line was obtained from the National Institutes of Health (NIH) AIDS Reagent Program, Division of AIDS, National Institute of Allergy and Infectious Diseases, NIH. Jurkat, Clone E6-1 and U937 were purchased from ATCC (TIB-152™ and CRL1593.2™) Jurkat 2D10 reporter cell line is described previously (Pearson, R., et al. J. Virol. 82: 12291-12303 (2008)). TZM-bl cells were cultured in DMEM high glucose complemented with 10% FBS and gentamicin (10 μg/ml). Jurkat and Jurkat 2D10 cells were cultured in RPMI medium containing 10% FBS and gentamicin (10 μg/ml). Primary culture of human astrocytes and microglia were obtained from the Tissue Culture Core facility of the Comprehensive NeuroAIDS Center (CNAC) in the Department of Neuroscience at the Lewis Katz School of Medicine at Temple University in Philadelphia.

Stable Cell Lines and Subcloning. TZM-bl cells were plated in 6 well plates at 1×10⁵ cells/well and transfected using Lipofectamine 2000 reagent (Invitrogen) with 1 μg of pX260-LTR_{(-80/+66)}-Cas9 plasmid. Next day cells were transferred into 100-mm dishes and cultured in the presence of puromycin (Sigma) at concentration 1 μg/ml. After two weeks surviving clones were isolated using cloning cylinders (Corning). Two million Jurkat 2D10 cells were electroporated with 10 μg pX260-LTR_{(-80/+66)}-Cas9 plasmid (Neon System, Invitrogen, 3 times 10 ms/1350V impulse). Forty-eight hours later medium was replaced with medium containing puromycin 0.5 μg/ml. After one week, selection puromycin was removed and cells were allowed to grow for another week. Next, cells were diluted to a concentration of 10 cells/ml plated in 96-well plates, 50 al/well and cultured for 2 weeks. Both TZM-bl and Jurkat 2D10 pX260-LTR_{(-80/+66)}-Cas9 single cell clones were screened for Cas9-FLAG expression after transfections with control pCMV-empty (pcDNA3.1) or pCMV-Tat plasmids by Western blot. Single cell clones with undetectable/very low level of Cas9 under control conditions and very high levels upon Tat overexpression were expanded and used in further experiments.

Lentivirus Packaging. HEK 293T cells were co-transfected using CaPO₄ precipitation method in the presence of chloroquine (50 μM) with packaging lentiviral vectors mixtures at 30 μg total DNA/2.5×10⁶ cells/100 mm dish. Next day medium was replaced and 24 and 48 h later supernatants were collected, clarified at 3000 RPM for 10 minutes, 0.45 μm filtered and concentrated by ultracentrifugation (2 h, 25000 RPMI, with 20% sucrose cushion). Lentiviral pellets were resuspended in HBSS by gentle agitation overnight, aliquoted and tittered in HEK 293T cells. Lenti-LTR_{(-80/+66)}Cas9-Blast lentivirus was tittered by FLAG immunocytochemistry, pKLV-U6-LTR A/B-PGKpuro2ABFP lentiviruses by BFP fluorescent microscopy.

Viral Stock. For creation of HIV-1_{NL4-3-EFGP-p2A-Nef}, fusion PCR (Heckman, K. L., et al. Nat. Protoc. 2, 924-932 (2007)) was used to amplify the EGFP gene, a P2A self-cleaving peptide (Kim, J. H., et al. PLoS One 6, e18556 (2011)), and N-terminal of HIV-1 Nef in frame with HIV-1 splicing acceptor originally for HIV-1 Nef expression. DNA was then cloned into the BamHI and XhoII restriction sites of the HIV-1 proviral clone pNL4-3 (Adachi, A., et al. J. Virol. 59, 284-291 (1986)). The self-cleaving P2A peptide from porcine teschovirus-1 between the GFP and Nef allows the expression of HIV-1 Nef in full length (Edmonds, T. G., et al. PBMC. Virology 408, 1-13 (2010)). In order to generate pEcoHIV-NL4-3-EGFP, the coding region of gp120 in HIV-1_{NL4-3} was replaced with gp80 from ecotropic murine leukemia PCR-amplified from pHCMV-EcoEnv (Sena-Esteves, M., et al. J. Virol. Methods 122, 131-139 2004; Addgene plasmid 15802), following the engineering strategy previously published (Potash, M. J., et al. Proc. Natl. Acad. Sci. USA 102, 3760-3765 (2005)).

HIV-1_{NL4-3-EGFP-P2A-Nef} reporter virus was prepared by transfecting HEK 293T cells with pNL4-3-EGFP-P2A-Nef plasmid (University of Pittsburgh School of Medicine) processed like lentiviral stocks (see above) and tittered by GFP-FACS in Jurkat cell line. HIV-1 JRFL and SF162 crude stocks used was prepared from supernatants of PBMCs infected with HIV-1 for 6 days, clarified at 3000RPM for 10 minutes and 0.45 μm filtered. Virus was tittered using Gag p24 ELISA.

In vitro HIV-1 Infection. Jurkat and U937 cells were infected by spinoculation for 2 h at 2700 RPM, 32° C. in 500 al inoculum containing 8 μg/ml polybrene then resuspended and left for 4 h, then 500 al of growth medium was added. The next day cells were washed 3 times with PBS and resuspended in growth medium. For infection of astrocytes and microglial cells, primary human fetal brain cells were transduced/infected by incubation with viral stocks diluted in Opti-MEM medium in the presence of polybrene (8 μg/ml) for 4 h, then 1 ml of growth medium was added for overnight. The next day cells were washed 3 times with PBS and fresh grow medium was added.

HIV-1 DNA Detection and Quantification. Genomic DNA was isolated from cells using NUCLEOSPIN Tissue kit (Macherey-Nagel Inc. Bethlehem, Pa.) according to the protocol of the manufacturer. For LTR specific PCRs (see Table 1; SEQ ID NOS: 7-14; forward (F) and reverse (R) β-actin—SEQ ID NOS: 15, 16 respectively), 100 ng of extracted DNA was subjected to PCR using FAILSAFE™ PCR kit and buffer D (Epicentre Technologies Corp., Madison, Wis.) under the following PCR conditions: 98° C. 5 minutes, 30 cycles (98° C. 30 s, 55° C. 30 s, 72° C. 30 s), 72° C. 7 minutes and resolved in 2% agarose gel. PCR products were subjected to agarose gel electrophoresis, gel purified, cloned into TA vector (Invitrogen Corp., Carlsbad, Calif.) and sent for Sanger sequencing (GENEWIZ Global, South Plainfield, N.J.). HIV-1 DNA was quantified using TAQMAN™ qPCR specific for HIV-1 5′-UTR and Env genes and cellular beta-globin gene as a reference (see Table 1, SEQ ID NOS: 17-25). Prior to qPCR, genomic DNA from infected cells was diluted to 10 ng/μl and then 5 μl (=50 ng) was taken per reaction/well. Reaction mixtures were prepared using Platinum TAQ DNA Polymerase (Invitrogen Corp., Carlsbad, Calif.) according to a simplified procedure (Liszewski, M. K., et al. Methods 47, 254-260 (2009)). Standard was prepared from serial dilutions of U1 cells genomic DNA since it contains two single copies of HIV-1 provirus per diploid genome equal to beta-globin gene copy number. qPCR conditions: 98° C. 5 minutes, 45 cycles (98° C. 15 s, 62° C. 30s with acquisition, 72° C. 1 minute). Reactions were carried out and data analyzed in a LIGHTCYCLER480™ (Roche Holding AG, Basel, Switzerland)).

Reverse Transcription and PCR. Total RNA was extracted from cells using RNEASY kit (Qiagen, Hilden, Germany) with on column DNAse I digestion. Next 1 μg of RNA was used for M-MLV reverse transcription reactions (Invitrogen Corp., Carlsbad, Calif.). cDNA was diluted and quantified using TAQMAN™ qPCR specific for HIV-1 Gag and Env genes and cellular beta-actin gene as a reference (Table 1, SEQ ID NOS: 17-25) under the same protocol like genomic DNA but analyzed using relative quantification mode.

Flow Cytometry. GFP expression in Jurkat 2D10 was quantified in live cells using GUAVA EASYCYTE Mini flow cytometer (Guava Technologies, Inc., Billerica, Mass.). HIV-1_{NL4-3-GFP-P2A-Nef} infected Jurkat cells were first fixed for 10 minutes in 2% paraformaldehyde then washed 3 times in PBS. Cell viability was assessed using propidium iodide staining. To 200 μl of live cells in suspension PI solution was added to final concentration 10 μg/ml. Samples were incubated for 5 minutes at room temperature in the dark. After incubation, samples were acquired using a GUAVA EASYCYTE Mini flow cytometer.

Western Blot. Whole cell lysates were prepared by incubation of Jurkat cells in TNN buffer [50 mM Tris pH 7.4, 150 mM NaCl, 1% Nonidet P-40, 5 mM EDTA pH 8, 1× protease inhibitor cocktail for mammalian cells (Sigma)] for 30 minutes on ice then pre-cleared by spinning at top speed for 10 minutes at 4° C. Fifty micrograms of lysates were denatured in 1× Laemli buffer and separated by SDS-polyacrylamide gel electrophoresis in Tris-glycine buffer followed by transfer onto nitrocellulose membrane (Bio-Rad Laboratories Inc., Hercules, Calif.). The membrane was blocked in 5% milk/PBST for 1 h and then incubated with mouse anti-flag M2 monoclonal antibody (1:1000, Sigma-Aldrich, St. Louis, Mo.) or mouse anti-α-tubulin monoclonal antibody (1:2000). After washing with PBST, the membranes were incubated with conjugated goat anti-mouse antibody (1:10,000) for 1 h at room temperature. The membrane was scanned and analyzed using an Odyssey Infrared Imaging System (LI-COR Biosciences, Lincoln, Nebr.).

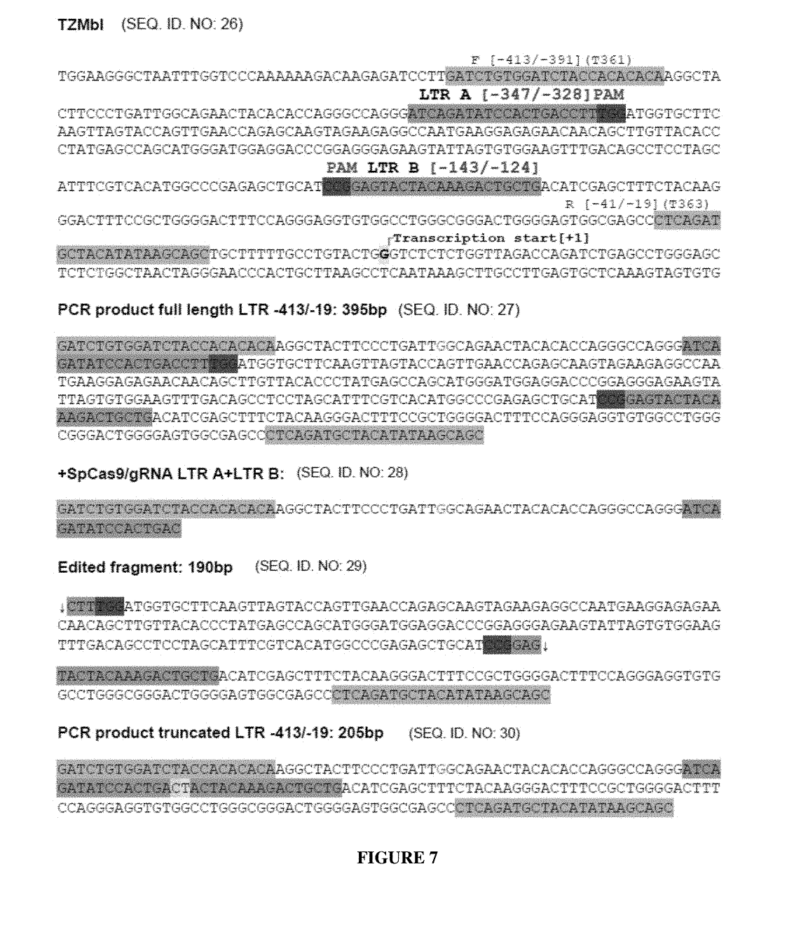

Results The coding DNA sequence corresponding to the Cas9 gene was placed in a pX260 expression vector, containing three different segments of the HIV-1 promoter spanning the U3 and R regions of the 5′-LTR to identify the minimal DNA elements of the viral promoter that remain responsive to Tat, yet lacks the sequences corresponding to gRNAs A and B that are initially used for editing HIV-1 DNA (FIG. 1A). After verification of this cloning strategy by DNA sequencing of each construct, expression of Cas9 by each vector and the level of their response to Tat was examined in TZM-bl cells co-transfected with pX260 or pX260-LTR-Cas9 and CMV-Tat. Results from Western blot revealed activation of Cas9 expression by all three constructs including the plasmid encompassing the minimal DNA promoter sequence positioned between −80 to +66 (FIG. 1B). This was particularly important for the studies as the promoter sequence resides outside of the DNA sequences corresponding to gRNAs A and B (FIG. 1B). Next, a DNA fragment corresponding to LTR_{(-80/+66)}-Cas9 was cloned into a lentiviral vector (LV) and used to transduce TZM-bl cells to assess the effect of Tat protein on the editing of integrated copies of HIV-1 DNA expressing the luciferase reporter gene. Results from PCR amplification of the LTR revealed the detection of 205 bp DNA fragment in cells expressing gRNAs A and B and Tat protein (FIG. 1C, compare lanes 1-5 to lanes 6-8). The position of the primers used for PCR amplification and the expected amplicons are illustrated in FIG. 1A (also see FIG. 7). Expression of Cas9, Tat and α-tubulin (control for equal loading) are shown in FIG. 1D.

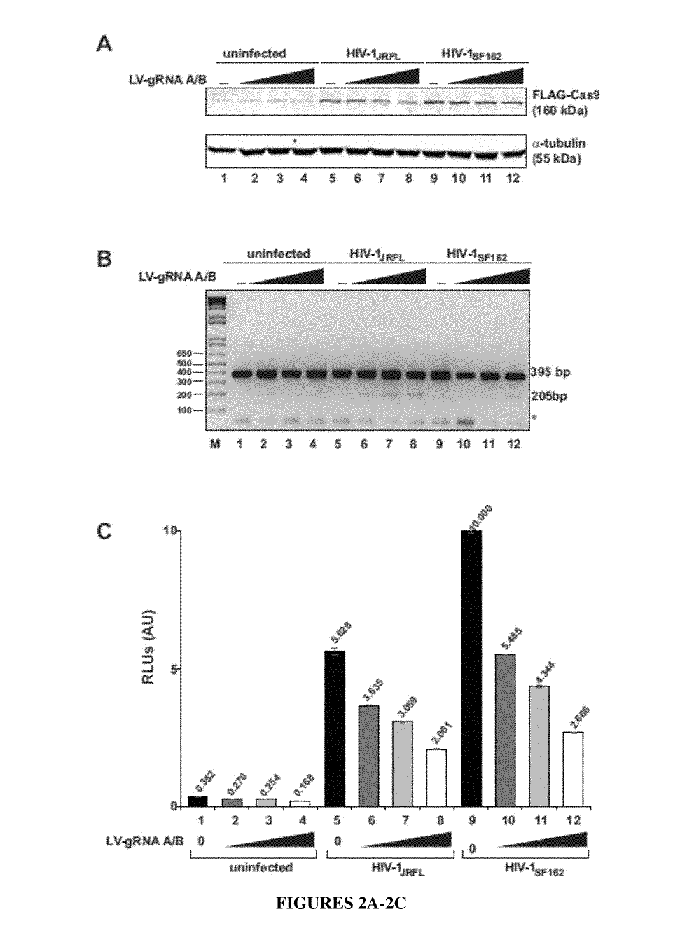

Next, the impact of the viral DNA excision on viral promoter activity by luciferase assay, was examined. Results showed a gradual decrease in luciferase activity upon activation of Cas9 by Tat, corroborating the results from DNA assay, indicating that the cleavage of DNA causes inhibition of viral promoter activity in these cells (FIG. 1E). In follow-up studies, the activation of Cas9 upon infection of TZMb1 cells by HIV-1 was investigated. To this end, the LTR_{(-84/+66)}-Cas9 reporter TZMb-1 cells were transduced by LV-gRNAs A/B for 24 hours, after which cells were infected with HIV-1_{JRFL} or HIV-1_{SF162} at three different MOIs. After 48 hours, cells were harvested for evaluating DNA excision by PCR, expression of the integrated promoter sequence by luciferase assay, and expression of Cas9 by Western blot. Results from these experiments show the detection of a post-cleavage 205 bp DNA fragment in cells infected with HIV-1_{JRFL} and HIV-1_{SF162}, indicating that production of Tat by HIV-1_{JRFL} and HIV-1_{SF162} transactivated the LTR_{(-80/+66)} promoter and production of Cas9 in these cells (FIG. 2A). Further, results from luciferase assay revealed significant reduction of luciferase activity in the cells, again verifying the effectiveness of Cas9 activation by Tat, which is produced upon infection by HIV-1_{JRFL} or HIV-1_{SF162} in shutting down the integrated HIV-1 luciferase gene. Induction of Cas9 in the infected cells is shown in FIG. 2B. Results from Western blot showed activation of the truncated LTR promoter, LTR_{(-80/+66)}, upon infection of cells with HIV-1JRFL and HIV-1SF162, resulting in the production of Cas9 protein in the cells (FIG. 2C).

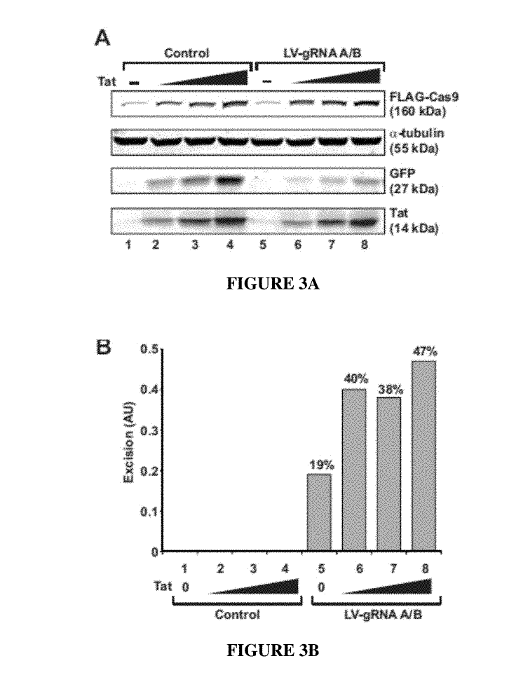

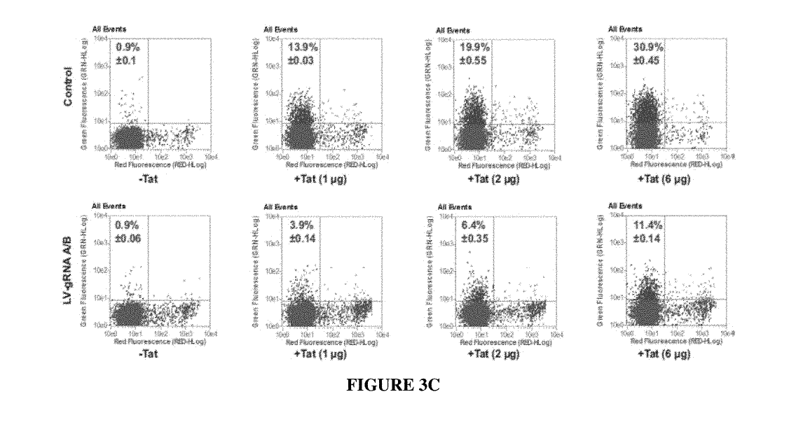

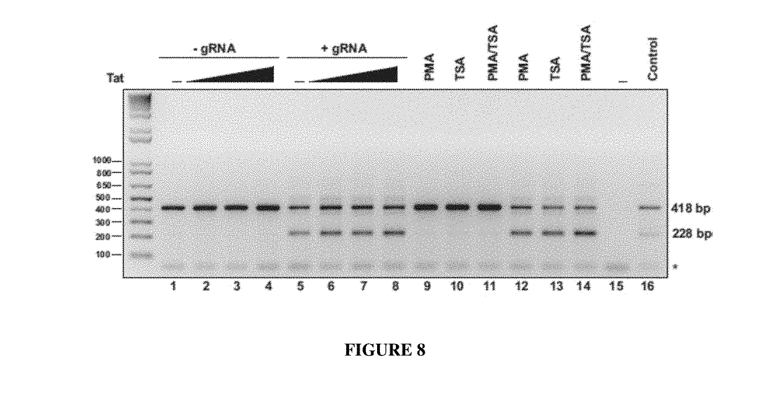

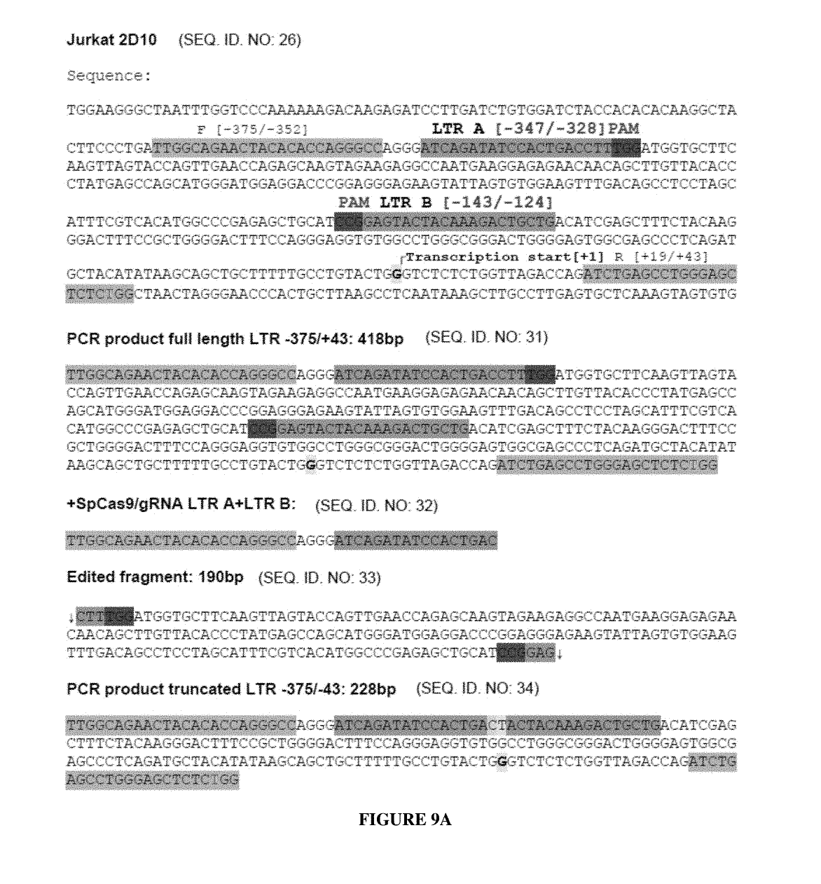

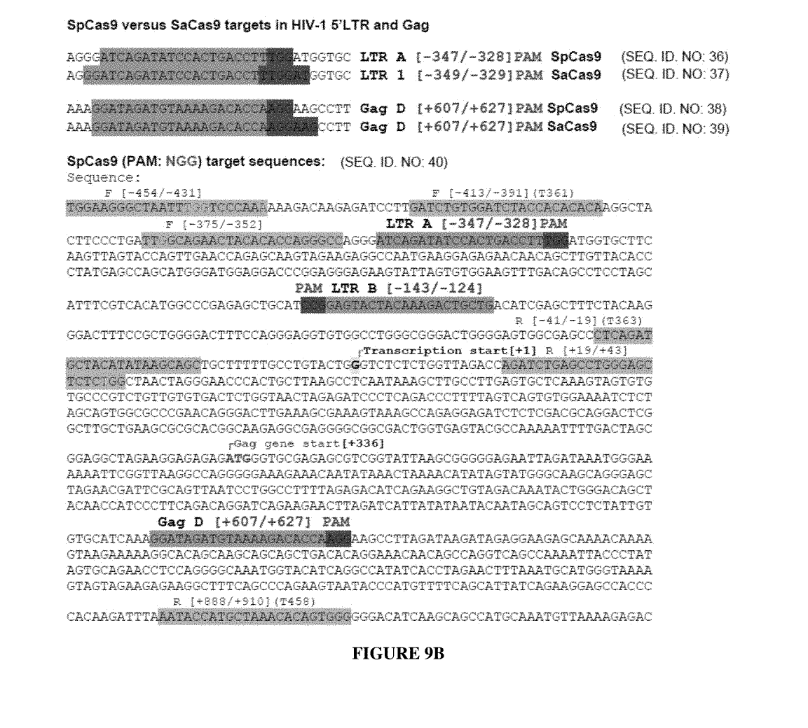

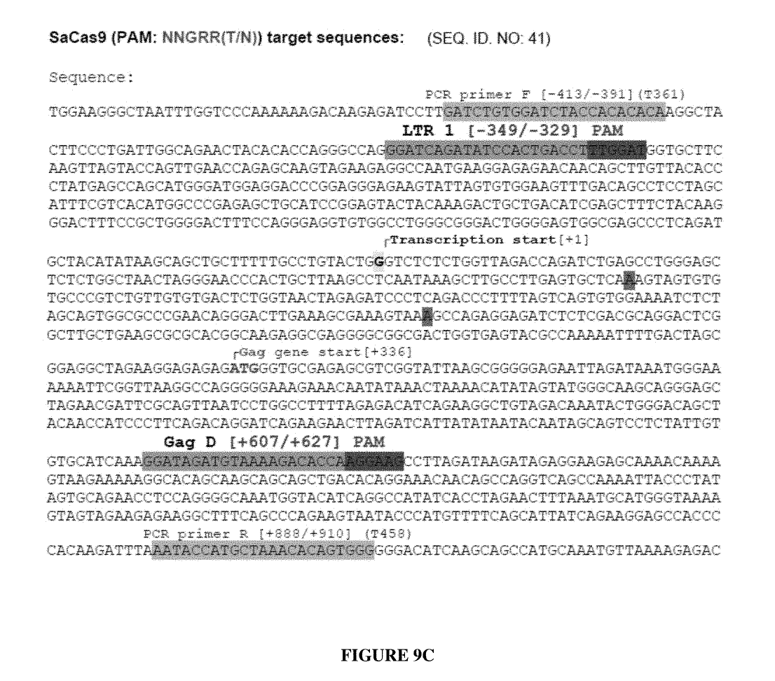

In a follow-up experiment, the ability of Tat-mediated activation of the LTR-Cas9 along with gRNAs A/B was tested in eliminating the HIV-1 genome in the human T-lymphocytic cells line, 2D10 (Pearson, R., et al. J. Virol. 82: 12291-12303 (2008)). These cells harbor integrated copies of a single round HIV-1_{NL4-3} in a latent state, whose genome lacks a portion of the Gag and Pol genes and the Nef gene is replaced by a gene encoding the reporter green fluorescent protein (GFP). The enhanced level of Tat protein in these cells and the activation of Cas9 (shown in FIG. 3A) caused editing of the viral LTR upon activation of Cas9 in the cells transduced by LV-gRNAs A/B (FIG. 3B, also see FIG. 8, lanes 1-8). Accordingly, a significant decrease in the number of GFP positive cells was detected in the presence of Tat (FIG. 3C), indicating that activation of Tat eliminates the capacity of the cleaved promoter in expressing viral DNA, which in turn, causes suppression of GFP in these cells. The DNA sequence corresponding to the position of the gRNAs, excision of the DNA fragment and PCR primers are shown in FIGS. 9A-9C, SEQ ID NOS: 26-40. The basal level of Cas9 expression and viral DNA excision may attribute to the constitutive but lowest expression of Tat in the latent 2D10 cell line.

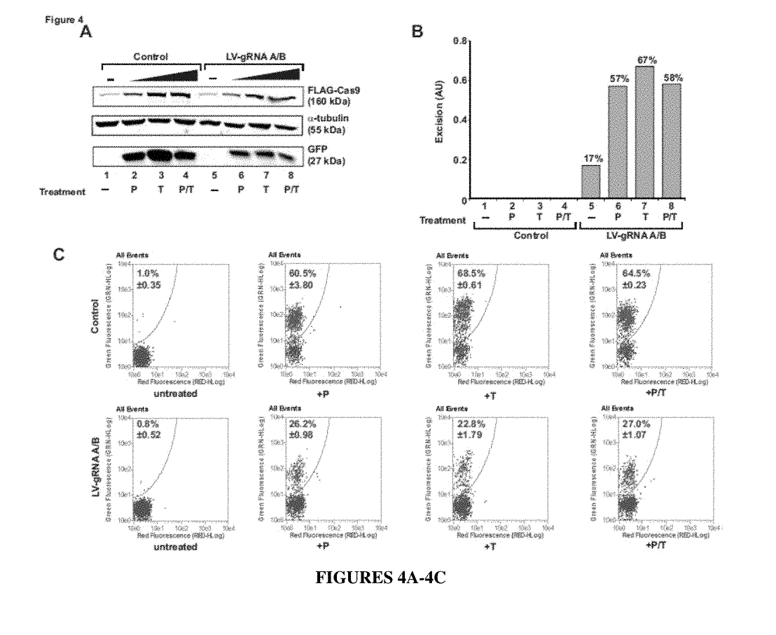

In light of earlier observations indicating the ability of PMA and/or TSA in stimulating integrated copies of proviral DNA in 2D10 cells (Pearson, R., et al. J. Virol. 82: 12291-12303 (2008)), the impact of PMA and TSA was assessed on the activation of Cas9 in a latently infected T-cell model. As seen in FIG. 4A, treatment of 2D10 cells with PMA and TSA, singly or in combination, increased the level of Cas9 expression. In a parallel experiment, PCR analysis was performed for the detection of LTR DNA and showed a clear increase in the level of viral DNA excision (FIG. 4B), as evidenced by the appearance of the 205 bp DNA fragment (see FIG. 8, lanes 9-14). Examination of viral activation by measuring the level of GFP in the cells using Western blot or the quantification of green fluorescent cells, indicative of viral activation, by fluorescent microscopy (FIG. 4C) showed a drastic decrease in the level of viral gene expression. Thus, it is likely that production of Cas9 upon activation of the minimal viral promoter (−88/+60) by either Tat, which is expressed upon reactivation of the silent provirus DNA or by PMA and TSA, leads to editing of the integrated copies of viral DNA and exerts a negative effect on the expression of the latent viral genome in cells containing gRNAs A and B.

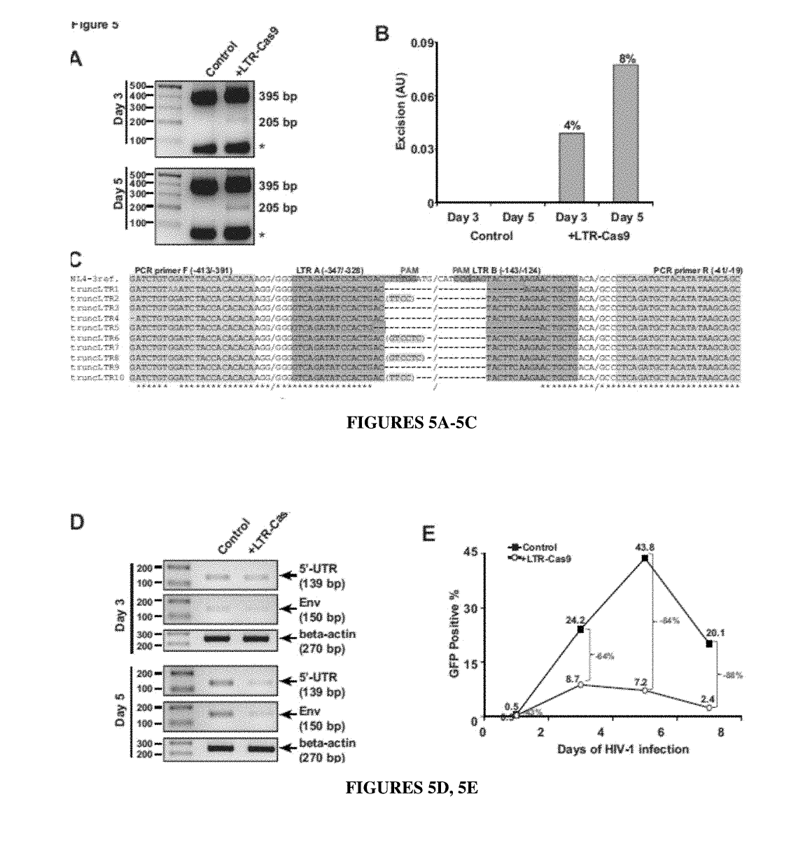

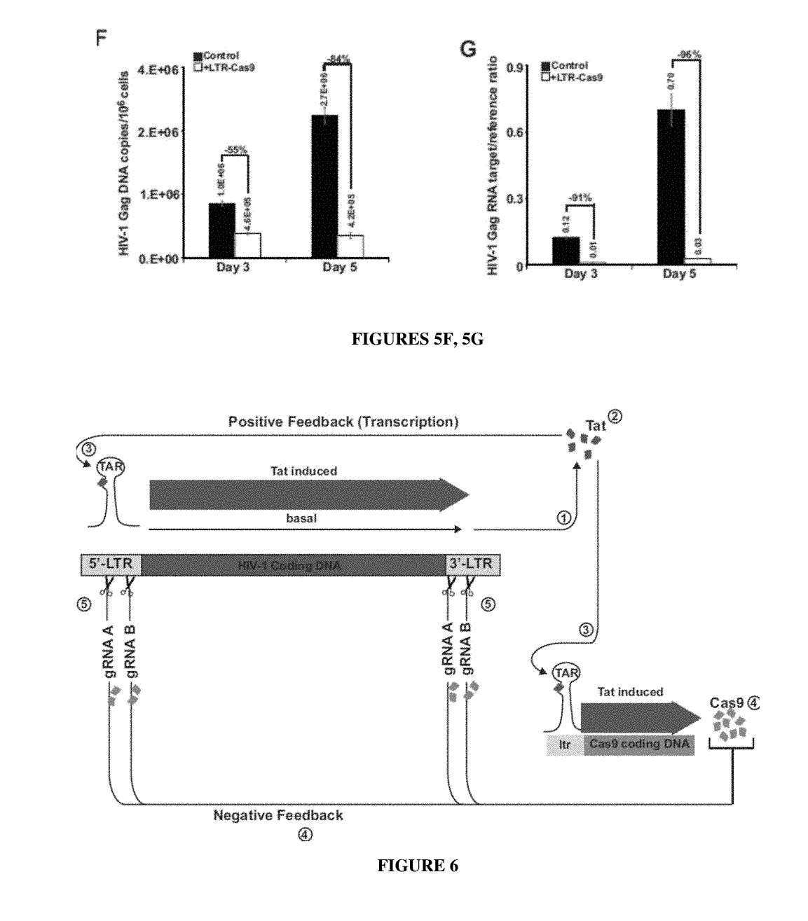

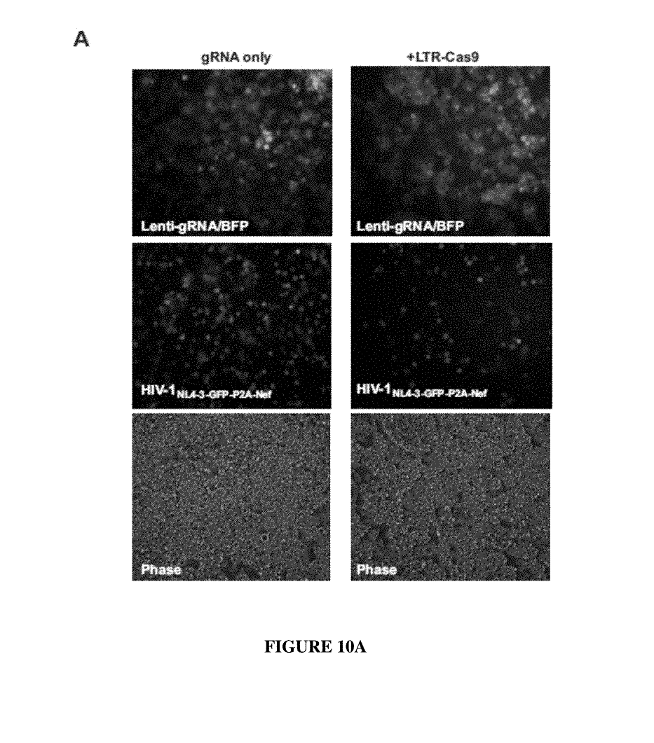

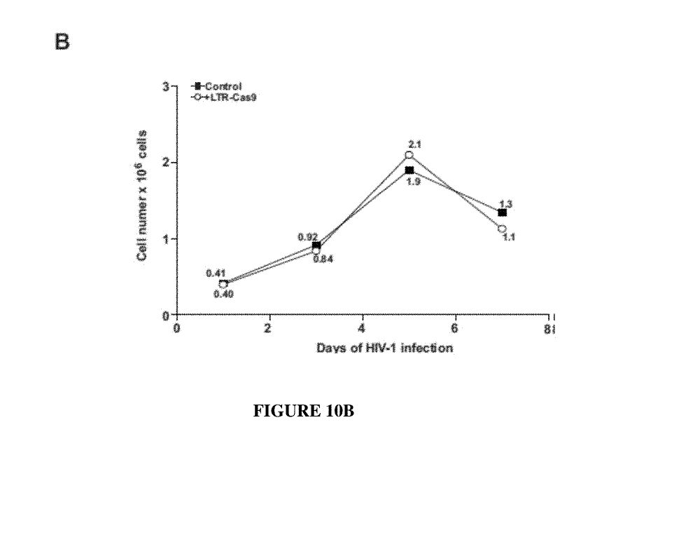

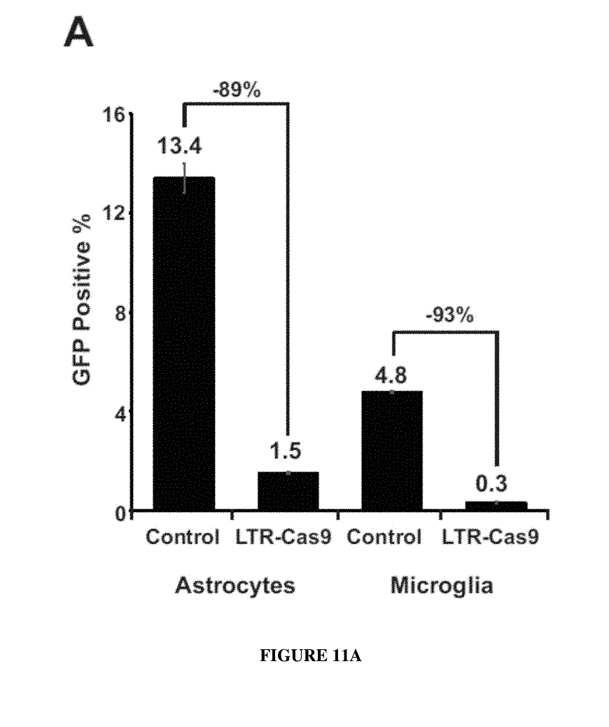

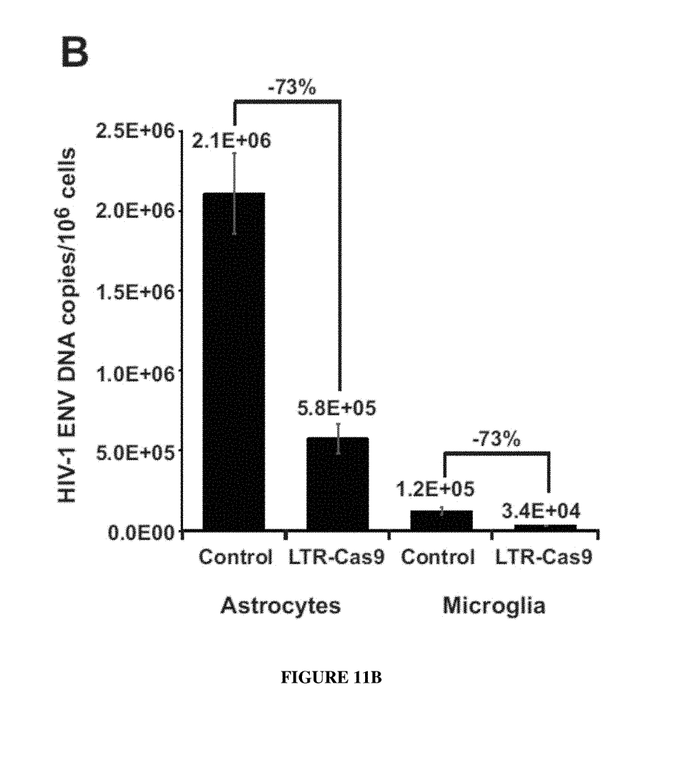

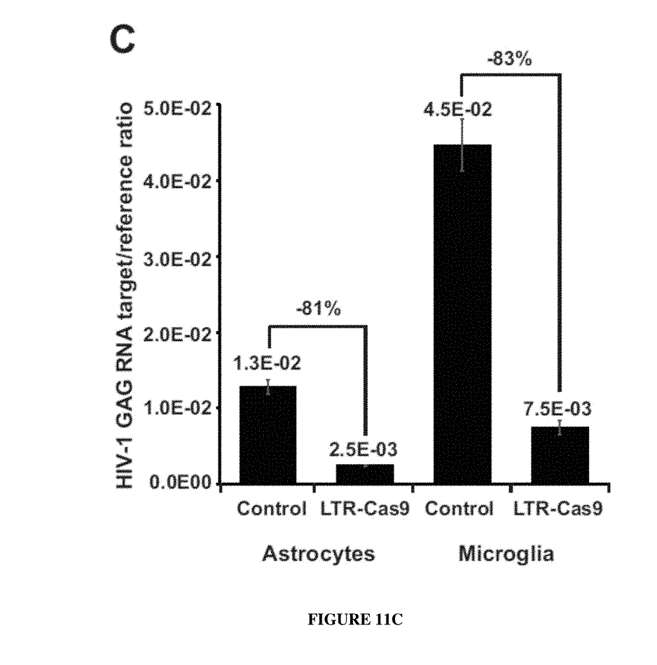

In the next series of experiments, the level of HIV-1 replication of Jurkat T-cells containing LTR-Cas9 was examined. Cells were transduced with lentivirus vector (LV) expressing gRNAs A and B, and LTR-88/+60-Cas9. After 24 hours, the transduced cells were infected with HIV-1_{NL4-3-EGFP-P2A-Nef}, and after 3 and 5 days, cells were harvested and viral DNAs were tested for the excision of a 190 bp DNA fragment spanning gRNAs A and B target sequences. As shown in FIGS. 5A and 5B), infection of cells with HIV-1 led to the appearance of a 205 bp amplicon in day 3 whose intensity was increased at day 5 of infection (FIGS. 5A and B). This observation provides evidence that, similar to the results shown in FIGS. 3A-3C, an increase in the level of Tat during the course of HIV-1 infection stimulated LTR-Cas9 expression, and hence, cleavage of LTR DNA. Direct sequencing of the 205 bp band seen in day 5 revealed cleavage sites within the LTR by Cas9/gRNA A and Cas9/gRNA B causing a range of InDel mutations that were detected in the junction of the 5′ and 3′ fusion sites (FIG. 5C). Examination of segments of the viral DNAs corresponding to the 5-UTR (nt+97 to +235) and envelop (env) gene (nt+5828 to +5977), both of which are positioned between the 5′ and 3′ LTRs, showed a substantial decrease in the intensity of a 139 bp and 150 bp amplicons corresponding to the 5′-UTR and env gene, respectively at day 5 compared to day 3 (FIG. 5D). These observations provide evidence that the excision of a larger DNA fragment of the HIV-1 genome spanning between the 5′ and 3′ LTRs upon cleavage by Cas9/gRNA A (at the 5′ LTR) and Cas9/gRNA B (at the 3′-LTR) may have also occurred upon treatment of the cells with Cas9 and gRNAs A and B, an event that has been reported previously (Hu, W., et al. Proc. Natl. Acad. Sci. USA 111, 11461-11466 (2014); Kaminski, R., et al. Sci. Rep. 6: 22555 (2016)). Quantitative analysis of the results from flow cytometry illustrating expression of the reporter GFP, indicative of viral gene activation, showed substantial inhibition of GFP positive cells (64%) on day 3 and even more on day 5 (84%) and day 8 (88%). The presence of lentivirus harboring genes encoding gRNAs and the marker BFP and expression of GFP in the cells were monitored by fluorescent microscopy and the quality of cell cultures was tested by phase microscopy (FIG. 10A). Quantitative analysis showed that the total number of cells remained unchanged, indicating that similar to the previous observation (Kaminski, R., et al. Sci. Rep. 6: 22555 (2016)), no toxicity is associated with this excision strategy. In accord with results from PCR gel analysis (shown in FIG. 5D), results from qPCR and qRT-PCR showed a significant decrease in the level of viral DNA copy numbers corresponding to the Gag gene, i.e. 55% on day 3 and 84% on day 5 and Gag RNA level 91% on day 3 and 96% on day 5 post infection (FIGS. 5F and 5G). A similar set of studies in human primary cultures of microglia and astrocytes was also performed. Results from these studies showed a significant suppression of viral gene expression and viral DNA presence in HIV-1 infected cells transduced with LVs expressing LTR-Cas9 and gRNAs (shown in FIGS. 11A-11C). Altogether, these observations provide evidence for the use of novel autoregulatory events by employing viral proteins, including Tat, to initiate the editing strategy using CRISPR/Cas9 by excising the viral genome and permanently suppressing viral replication.

Discussion

Since its discovery in 1985 (Arya, S. K., et al. Science 229: 69-73 (1985); Sodroski, J., et al. Science 227, 171-173 (1985)), the Tat transactivator protein of HIV-1 has been shown to be a critical regulatory protein due to its role in expression of the viral genome at the transcriptional level and its pathogenic impact on uninfected cells. Mechanistically, Tat associates with the RNA sequence located downstream of the initiation site from transcription (nucleotides+1 to +59), the so-called transcription responsive region or TAR. The association of Tat with TAR triggers a series of molecular and biochemical events leading to the formation of pre-initiation and initiation complexes of transcription in proximity to the transcription start site (nucleotide+1). This complex includes a series of cellular proteins that have the ability to phosphorylate or acetylate components of the complexes including pTEF and RNA polymerase II, thus facilitating transcriptional elongation of RNA (for review see Mbonye, U., et al. Virology 454-455, 328-339 (2014); Taube. R., et al. Viruses 5, 902-927 (2013)). In addition, the interaction of Tat with various transcriptional factors including NF-κB (Taylor, J.P., Khalili, K. Adv. Neuroimmunol. 4, 291-303 (1994)), p300/CBP and GCN5 (Colm E., et al. J. Biol. Chem. 276, 28179-28184 (2001); Kiernan, R. E., et al. EMBO J. 18, 6106-6118 (1999); Ott, M., et al. Curr. Biol. 9, 1489-1492 (1999)) can affect transcription of other viral and cellular genes; all of which contribute to the disease spectrum seen in HIV-1 positive AIDS patients (Gibellini, D., et al. New Microbiol. 28, 95-109 (2005)). Tat also plays a major role in the productive replication of latent virus in reservoirs once transcription from the reactivated viral promoter leads to an initial round of viral transcription and Tat production. The unique importance of Tat in HIV-1 replication and the pathogenesis of AIDS, provided a strong rationale for serving as a potential target for drug discovery as well as vaccine development. In fact, several potent inhibitors, some with the ability to interfere with Tat-TAR interaction and others with the capacity to prevent Tat communication with its cellular partners, have shown various degrees of efficacy in affecting HIV-1 replication (Tabarrini. O., et al. Future Med Chem 8, 421-442 (2016)). The strategy that was utilized in this study was to recruit Tat to stimulate Cas9 expression and promote excision of a segment of the viral genome and permanently ablate HIV-1 gene transcription and replication in cells with productive or latent HIV-1. Here a suicide path for HIV-1 was designed so that it is triggered by Tat and includes editing of the viral genome using CRISPR/Cas9 technology (illustrated in FIG. 6). According to this pathway, production of Tat in the cells, in addition to stimulating its own promoter with the full-length 5′-LTR sequence, potentiates expression of Cas9 through the same mechanism by a truncated minimal promoter sequence spanning the GC-rich, TATA box, and TAR (−80 to +66) regions. Production of Cas9 and its association with gRNAs designed to target the LTR DNA sequence outside of the (−80 to +66) induced InDel mutations within the full-length viral promoter and by excising a segment of the gene, can permanently eradicate HIV-1 in the cells. In addition to the expected 417 bp DNA fragment representing the full-length LTR sequence, results from short-range amplification of LTR DNA showed a second DNA fragment of 227 bp in size found only in cells expressing Tat. The 227 bp DNA fragment was generated by joining the residual 5′-LTR to the remaining 3′-LTR after cleavage by Cas9/gRNA A at either the 5′-LTR or the 3′-LTR. It is also likely that ligation of the remaining DNA fragment from the 5′-LTR with those from the 3′-LTR after cleavage by Cas9/gRNA created a new template for gene amplification and the appearance of a similar size (227 bp) amplicon as reported previously (Kaminski, R., et al. Sci. Rep. 6: 22555 (2016)).

The CRISPR/Cas9 gene editing strategy has received attention in biomedical research in recent years due to its extraordinary ability to edit the genome with precision and high efficiency and its simplicity and flexibility of implementation. However, there are several areas that need close attention. For example, it is important to design the most specific and effective gRNAs to avoid off-target effects. The strategy that was developed here, has been employed for maximizing specificity and avoiding off-target editing which was verified by ultra-deep sequencing of the whole genome and various other tests, as described (Hu, W., et al. Proc. Natl. Acad. Sci. USA 111, 11461-11466 (2014); Kaminski, R., et al. Sci. Rep. 6: 22555 (2016)). Treatment of the cells by a single RNA may lead to the development of mutant HIV-1 as a result of unfaithful NHEJ repair at the site of cleavage, and potentially lead to the emergence of mutant virus that becomes resistant to the initial single gRNA (Wang, G., et al. Mol. Ther. 24, 522-526 (2016a); Wang, Z., et al. Cell Rep. 15, 481-489 (2016b)). Employment of multiplex of gRNAs, which, by introducing multiplex double-strand breaks across the HIV-1 genome, leads to the excision of a larger segment of viral DNA from the host genome, alleviating this concern and permanently eliminating any chance for the emergence of replication-competent virus (Khalili, K., et al. J. Neurovirol. 21, 310-321 (2015); Hu, W., et al. Proc. Natl. Acad. Sci. USA 111, 11461-11466 (2014); Kaminski, R., et al. Sci. Rep. 6: 22555 (2016); Ebina, H., et al. Sci. Rep. 3, 2510 (2013); Liao, H. K., et al. Nature Comm. 6, 6413 (2015)). The second issue relates to the controlled expression of Cas9 to avoid the unnecessary persistence of expression of the protein that may non-specifically cause injury to the host genome in the long term and/or induce an immune response. The strategy herein for conditional expression of Cas, by HIV-1 Tat, would provide a novel approach for stimulating the silent gene editing molecule to be expressed and to excise HIV-1 DNA at the early stage of virus reactivation.

TABLE 1

| primer | sequence |
|---|---|
| 1. Cloning pX260-LTR- | |
| Cas9 constructs | |
| Kpn1-LTR(−454)-F | 5′-GGTACCTGGAAGGGCTAATTTGG-3′ (SEQ ID NO: 1) |
| Kpn1-LTR(−120)-F | 5′-GGTACCTCGAGCTTTCTACAAGG-3′ (SEQ ID NO: 2) |
| Xba1-LTR(−80)-F | 5′-TCTAGAGGAGGTGTGGCCTGGGC-3′ (SEQ ID NO: 3) |
| LTR(+66)-Nco1-R | 5′-CCATGGTAAGCAGTGGGTTCC-3′ (SEQ ID NO: 4) |
| 2. Cloning lentiLTR | |
| (−80/+66)-Cas9- | |
| Blast construct | |
| Nhe1-LTR(−80)-F | 5′-GCTAGCGGAGGTGTGGCCTGGGC-3′ (SEQ ID NO: 5) |
| LTR(+66)-Xba1-R | 5′-TCTAGATAAGCAGTGGGTTCC-3′ (SEQ ID NO: 6) |
| 3. PCRs | |
| LTR −417/F | 5′-GATCTGTGGATCTACCACACACA-3′ (SEQ ID NO: 7) |
| LTR −19/R | 5′-GCTGCTTATATGTAGCATCTGAG-3′ (SEQ ID NO: 8) |
| LTR −374/F | 5′-TTAGCAGAACTACACACCAGGGCC-3′ (SEQ ID NO: 9) |
| LTR +43/R | 5′-CCGAGAGCTCCCAGGCTCAGATCT-3′ (SEQ ID NO: 10) |
| HIV-1 5′UTR +97/F | 5′-AAGTAGTGTGTGCCCGTCTG-3′ (SEQ ID NO: 11) |
| HIV-1 5′UTR +235/R | 5′-TCGAGAGATCTCCTCTGGCT-3′ (SEQ ID NO: 12) |
| HIV-1 Env +5828/F | 5′-TCCTTGGGATGTTGATGATCT-3′ (SEQ ID NO: 13) |
| HIV-1 Env +5977/R | 5′-TGGCCCAAACATTATGTACC-3′ (SEQ ID NO: 14) |
| b-actin/F | 5′-CTACAATGAGCTGCGTGTGGC-3′ (SEQ ID NO: 15) |
| b-actin/R | 5′-CAGGTCCAGACGCAGGATGGC-3′ (SEQ ID NO: 16) |
| 4. Taqman qPCRs | |
| HIV-1 5′UTR F | 5′-AAGTAGTGTGTGCCCGTCTG-3′ (SEQ ID NO: 17) |
| HIV-1 5′UTR R | 5′-TCGAGAGATCTCCTCTGGCT-3′ (SEQ ID NO: 18) |
| HIV-1 5′UTR Probe | 5′-FAM-CTGTTCGGGCGCCACTGCTA-ZEN-IowaBlackFQ-3′ |
| | (SEQ ID NO: 19) |
| Hs b-globin F | 5′-CCCTTGGACCCAGAGGTTCT-3′ (SEQ ID NO: 20) |
| Hs b-globin R | 5′-CGAGCACTTTCTTGCCATGA-3′ (SEQ ID NO: 21) |
| Hs b-globin probe: | 5′-FAM-GCGAGCATCTGTCCACTCCTGATGCTGTTATGGGCGCT |
| | CGC-ZEN-IowaBlackFQ-3′ (SEQ ID NO: 22) |
| Hs b-actin F | 5′-TGGACTTCGAGCAAGAGATG-3′ (SEQ ID NO: 23) |
| Hs b-actin R | 5′-GAAGGAAGGCTGGAAGAGTG-3′ (SEQ ID NO: 24) |
| Hs b-actin probe: | 5′-FAM-CGGCTGCTTCCAGCTCCTCC-ZEN-IowaBlackFQ-3′ |
| | (SEQ ID NO: 24) |

What is claimed:

1. A pharmaceutical composition comprising: an isolated nucleic acid sequence encoding a clustered regularly interspaced short palindromic repeats (CRISPR)-associated endonuclease operably linked to a minimal human immunodeficiency virus (HIV) long terminal repeat (LTR) promoter comprising a trans-activator of transcription (Tat) responsive element of the HIV LTR promoter; and/or,
at least one isolated nucleic acid encoding at least one guide RNA, wherein the at least one guide RNA is complementary to a target nucleic acid sequence in HIV.

2. The pharmaceutical composition of claim 1, wherein the minimal human immunodeficiency virus (HIV) long terminal repeat (LTR) promoter further comprises a core region.

3. The pharmaceutical composition of claim 1, wherein the minimal human immunodeficiency virus (HIV) long terminal repeat (LTR) promoter comprises a nucleic acid sequence having at least about a 75% sequence identity to nucleic acid sequences from about position −120 up to about a position +66.

4. The pharmaceutical composition of any one of claims 1-3, wherein the minimal human immunodeficiency virus (HIV) long terminal repeat (LTR) promoter comprises nucleic acids from about position −120 up to about a position +66.

5. The pharmaceutical composition of any one of claims 1-4, wherein the minimal human immunodeficiency virus (HIV) long terminal repeat (LTR) promoter comprises nucleic acids from about position −80 up to about a position +66.

6. The pharmaceutical composition of claim 1, wherein the target nucleic acid sequence in HIV comprises a sequence within a coding region or a non-coding region of HIV.

7. The pharmaceutical composition of claim 6, wherein the non-coding region comprises a long terminal repeat of HIV or a sequence within the long terminal repeat of HIV.

8. The pharmaceutical composition of claim 7, wherein the sequence within the long terminal repeat of HIV comprises a sequence within the U3, R, or U5 regions that excludes any sequence of the minimal HIV LTR promoter.

9. The pharmaceutical composition of any one of claims 1-8, further comprising a plurality of guide RNA nucleic acid sequences complementary to a plurality of target nucleic acid sequences of HIV.

10. The pharmaceutical composition of any one of claims 1-9, wherein the CRISPR-associated endonuclease is Cas9.

11. The pharmaceutical composition of any one of claims 1-10, wherein the CRISPR-associated endonuclease is optimized for expression in a human cell.

12. The pharmaceutical composition of any one of claims 1-11, further comprising a sequence encoding a transactivating small RNA (tracrRNA), wherein the tracrRNA is fused to a sequence encoding a guide RNA.

13. The pharmaceutical composition of any one of claims 1-12, wherein the isolated nucleic acid sequence is operably linked to an expression vector comprising: a lentiviral vector, an adenoviral vector, or an adeno-associated virus vector.

14. An isolated nucleic acid sequence comprising a sequence encoding a clustered regularly interspaced short palindromic repeats (CRISPR)-associated endonuclease operably linked to a minimal human immunodeficiency virus (HIV) long terminal repeat (LTR) promoter comprising a trans activation response element (TAR) of the HIV LTR promoter.

15. The isolated nucleic acid sequence of claim 14, wherein the minimal human immunodeficiency virus (HIV) long terminal repeat (LTR) promoter further comprises a core region.

16. The isolated nucleic acid sequence of claim 15, wherein the minimal human immunodeficiency virus (HIV) long terminal repeat (LTR) promoter comprises a nucleic acid sequence having at least about a 75% sequence identity to nucleic acid sequences from about position −120 up to about a position +66.

17. The isolated nucleic acid sequence of claim 15, wherein the minimal human immunodeficiency virus (HIV) long terminal repeat (LTR) promoter comprises nucleic acids from about position −120 up to about a position +66.

18. The isolated nucleic acid sequence of claim 15, wherein the minimal human immunodeficiency virus (HIV) long terminal repeat (LTR) promoter comprises nucleic acids from about position −80 up to about a position +66.

19. The isolated nucleic acid sequence of any one of claims 14-18, wherein the CRISPR-associated endonuclease is Cas9.

20. The isolated nucleic acid sequence of any one of claims 14-19, wherein the CRISPR-associated endonuclease is optimized for expression in a human cell.

21. The isolated nucleic acid sequence of any one of claims 14-20, wherein the isolated nucleic acid sequence is operably linked to an expression vector, wherein the expression vector comprises: a lentiviral vector, an adenoviral vector, or an adeno-associated virus vector.

22. A method of treating a subject having a human immunodeficiency virus (HIV) infection, the method comprising:
administering to the subject a composition comprising an isolated nucleic acid sequence encoding a clustered regularly interspaced short palindromic repeats (CRISPR)-associated endonuclease operably linked to a minimal human immunodeficiency virus (HIV) long terminal repeat (LTR) promoter comprising a trans-activator of transcription (Tat) responsive element of the HIV LTR promoter; and/or,
at least one isolated nucleic acid encoding at least one guide RNA, wherein the at least one guide RNA is complementary to a target nucleic acid sequence in an HIV genome.

23. The method of claim 22, wherein HIV infection is an active or latent infection.

24. The method of claim 22, wherein the pharmaceutical composition is administered topically or parenterally.

25. The method of claim 22, wherein the minimal human immunodeficiency virus (HIV) long terminal repeat (LTR) promoter further comprises a core region.

26. The method of claim 22, wherein the minimal human immunodeficiency virus (HIV) long terminal repeat (LTR) promoter comprises a nucleic acid sequence having at least about a 75% sequence identity to nucleic acid sequences from about position −120 up to about a position +66.

27. The method of any one of claims 22-26, wherein the minimal human immunodeficiency virus (HIV) long terminal repeat (LTR) promoter comprises nucleic acids from about position −120 up to about a position +66.

28. The method of any one of claims 22-27, wherein the minimal human immunodeficiency virus (HIV) long terminal repeat (LTR) promoter comprises a nucleic acid sequence having at least about a 75% sequence identity to nucleic acid sequences from about position −80 up to about a position +66.

29. The method of any one of claims 22-28, wherein the minimal human immunodeficiency virus (HIV) long terminal repeat (LTR) promoter comprises nucleic acids from about position −80 up to about a position +66.

30. The method of any one of claims 22-29, wherein the CRISPR-associated endonuclease is Cas9.

31. The method of any one of claims 22-30, wherein the CRISPR-associated endonuclease is optimized for expression in a human cell.

32. The method of any one of claims 22-31, wherein an expression vector comprises the isolated nucleic acid sequence encoding the CRISPR-associated endonuclease operably linked to the minimal human immunodeficiency virus (HIV) long terminal repeat (LTR) promoter and at least one isolated nucleic acid encoding at least one guide RNA, wherein the at least one guide RNA is complementary to a target nucleic acid sequence in an HIV genome.

33. The method of any one of claims 22-32, wherein a first expression vector comprises the isolated nucleic acid sequence encoding the CRISPR-associated endonuclease operably linked to the minimal human immunodeficiency virus (HIV) long terminal repeat (LTR) promoter and a second expression vector comprises the at least one isolated nucleic acid encoding at least one guide RNA, wherein the at least one guide RNA is complementary to a target nucleic acid sequence in an HIV genome

34. The method of claim 33, wherein the first and second expression vectors are co-expressed in the host cell in vitro or in vivo.

35. The method of claim 32 or 33, wherein the expression vector comprises: a lentiviral vector, an adenoviral vector, or an adeno-associated virus vector.

36. The method of claim 32 or 33, wherein an expression vector encodes a plurality of guide RNAs and/or a plurality of expression vectors each encode one or more guide RNAs.

37. The method of any one of claims 22-37, further comprising administering one or more Tat activators, anti-viral agents or combinations thereof.

38. An expression vector for eradicating a human immunodeficiency virus (HIV) nucleic acid sequence integrated into the genome of an in vitro or in vivo host cell latently infected with HIV, wherein the expression vector comprises at least one isolated nucleic acid sequence encoding a Clustered Regularly Interspaced Short Palindromic Repeat (CRISPR)-associated endonuclease operably linked to a minimal HIV long terminal repeat (LTR) promoter comprising a trans-activator of transcription (Tat) responsive element of the HIV LTR promoter; and/or,
at least one guide RNA (gRNA), the gRNA being complementary to a target sequence in a proviral DNA for, eradicating HIV integrated into the genome of a latently infected host cell.

39. The expression vector of claim 38, wherein the gRNA nucleic acid sequence includes at least a first guide gRNA that is complementary to a first target sequence in a proviral DNA; and a second gRNA that is complementary to a second target sequence in the proviral DNA.

40. The expression vector of claim 38, wherein the minimal human immunodeficiency virus (HIV) long terminal repeat (LTR) promoter comprises a nucleic acid sequence having at least about a 75% sequence identity to nucleic acid sequences from about position −80 up to about a position +66.

41. The expression vector of any one of claims 38-40, wherein the minimal human immunodeficiency virus (HIV) long terminal repeat (LTR) promoter comprises nucleic acids from about position −80 up to about a position +66.

42. An isolated nucleic acid sequence encoding a clustered regularly interspaced short palindromic repeats (CRISPR)-associated endonuclease (CRISPR/Cas) operably linked to a minimal functional viral promoter whereby the minimal viral promoter is under control of an immediate early transcriptional activator.

43. A composition comprising an isolated nucleic acid sequence encoding a clustered regularly interspaced short palindromic repeats (CRISPR)-associated endonuclease (CRISPR/Cas) operably linked to a minimal functional viral promoter whereby the minimal viral promoter is under control of an immediate early transcriptional activator; and/or,
an isolated nucleic acid comprising at least one guide RNA that is complementary to a target nucleic acid sequence in the virus.

44. The composition of 42, further comprising an expression vector encoding the isolated nucleic acid sequence comprising the CRISPR-associated endonuclease operably linked to the minimal virus promoter and at least one isolated nucleic acid encoding at least one guide RNA, wherein the at least one guide RNA is complementary to a target nucleic acid sequence in virus genome.

45. The composition of claim 42, wherein a first expression vector comprises the isolated nucleic acid sequence comprising the CRISPR-associated endonuclease operably linked to the minimal virus HIV long terminal repeat (LTR) promoter and a second expression vector comprising at least one isolated nucleic acid encoding at least one guide RNA, wherein the at least one guide RNA is complementary to a target nucleic acid sequence in the viral genome.


SEQUENCE LISTING (85 sequences)

SEQ ID NO: 1 - DNA, 23 bp, Artificial Sequence
  Description of Artificial Sequence: Synthetic primer
ggtacctggaagggctaatttgg

SEQ ID NO: 2 - DNA, 23 bp, Artificial Sequence
  Description of Artificial Sequence: Synthetic primer
ggtacctcgagctttctacaagg

SEQ ID NO: 3 - DNA, 23 bp, Artificial Sequence
  Description of Artificial Sequence: Synthetic primer
tctagaggaggtgtggcctgggc

SEQ ID NO: 4 - DNA, 21 bp, Artificial Sequence
  Description of Artificial Sequence: Synthetic primer
ccatggtaagcagtgggttcc

SEQ ID NO: 5 - DNA, 23 bp, Artificial Sequence
  Description of Artificial Sequence: Synthetic primer
gctagcggaggtgtggcctgggc

SEQ ID NO: 6 - DNA, 21 bp, Artificial Sequence
  Description of Artificial Sequence: Synthetic primer
tctagataagcagtgggttcc

SEQ ID NO: 7 - DNA, 23 bp, Artificial Sequence
  Description of Artificial Sequence: Synthetic primer
gatctgtggatctaccacacaca

SEQ ID NO: 8 - DNA, 23 bp, Artificial Sequence
  Description of Artificial Sequence: Synthetic primer
gctgcttatatgtagcatctgag

SEQ ID NO: 9 - DNA, 24 bp, Artificial Sequence
  Description of Artificial Sequence: Synthetic primer
ttagcagaactacacaccagggcc

SEQ ID NO: 10 - DNA, 24 bp, Artificial Sequence
  Description of Artificial Sequence: Synthetic primer
ccgagagctcccaggctcagatct

SEQ ID NO: 11 - DNA, 20 bp, Artificial Sequence
  Description of Artificial Sequence: Synthetic primer
aagtagtgtgtgcccgtctg

SEQ ID NO: 12 - DNA, 20 bp, Artificial Sequence
  Description of Artificial Sequence: Synthetic primer
tcgagagatctcctctggct

SEQ ID NO: 13 - DNA, 21 bp, Artificial Sequence
  Description of Artificial Sequence: Synthetic primer
tccttgggatgttgatgatct

SEQ ID NO: 14 - DNA, 20 bp, Artificial Sequence
  Description of Artificial Sequence: Synthetic primer
tggcccaaacattatgtacc

SEQ ID NO: 15 - DNA, 21 bp, Artificial Sequence
  Description of Artificial Sequence: Synthetic primer
ctacaatgagctgcgtgtggc

SEQ ID NO: 16 - DNA, 21 bp, Artificial Sequence
  Description of Artificial Sequence: Synthetic primer
caggtccagacgcaggatggc

SEQ ID NO: 17 - DNA, 20 bp, Artificial Sequence
  Description of Artificial Sequence: Synthetic primer
aagtagtgtgtgcccgtctg

SEQ ID NO: 18 - DNA, 20 bp, Artificial Sequence
  Description of Artificial Sequence: Synthetic primer
tcgagagatctcctctggct

SEQ ID NO: 19 - DNA, 20 bp, Artificial Sequence
  Description of Artificial Sequence: Synthetic probe
ctgttcgggcgccactgcta

SEQ ID NO: 20 - DNA, 20 bp, Artificial Sequence
  Description of Artificial Sequence: Synthetic primer
cccttggacccagaggttct

SEQ ID NO: 21 - DNA, 20 bp, Artificial Sequence
  Description of Artificial Sequence: Synthetic primer
cgagcactttcttgccatga

SEQ ID NO: 22 - DNA, 41 bp, Artificial Sequence
  Description of Artificial Sequence: Synthetic probe
gcgagcatctgtccactcctgatgctgttatgggcgctcgc

SEQ ID NO: 23 - DNA, 20 bp, Artificial Sequence
  Description of Artificial Sequence: Synthetic primer
tggacttcgagcaagagatg

SEQ ID NO: 24 - DNA, 20 bp, Artificial Sequence
  Description of Artificial Sequence: Synthetic primer
gaaggaaggctggaagagtg

SEQ ID NO: 25 - DNA, 20 bp, Artificial Sequence
  Description of Artificial Sequence: Synthetic probe
cggctgcttccagctcctcc

SEQ ID NO: 26 - DNA, 560 bp, Human immunodeficiency virus 1
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtg

SEQ ID NO: 27 - DNA, 395 bp, Human immunodeficiency virus 1
gatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagc

SEQ ID NO: 28 - DNA, 83 bp, Human immunodeficiency virus 1
gatctgtggatctaccacacacaaggctacttccctgattggcagaactacacaccagggccagggatcagatatccactgac

SEQ ID NO: 29 - DNA, 312 bp, Human immunodeficiency virus 1
ctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagc

SEQ ID NO: 30 - DNA, 205 bp, Human immunodeficiency virus 1
gatctgtggatctaccacacacaaggctacttccctgattggcagaactacacaccagggccagggatcagatatccactgactactacaaagactgctgacatcgagctttctacaagggactttccgctggggactttccagggaggtgtggcctgggcgggactggggagtggcgagccctcagatgctacatataagcagc

SEQ ID NO: 31 - DNA, 418 bp, Human immunodeficiency virus 1
ttggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctgg

SEQ ID NO: 32 - DNA, 45 bp, Human immunodeficiency virus 1
ttggcagaactacacaccagggccagggatcagatatccactgac

SEQ ID NO: 33 - DNA, 190 bp, Human immunodeficiency virus 1
ctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggag

SEQ ID NO: 34 - DNA, 228 bp, Human immunodeficiency virus 1
ttggcagaactacacaccagggccagggatcagatatccactgactactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctgg

SEQ ID NO: 35 - skipped

SEQ ID NO: 36 - DNA, 34 bp, Human immunodeficiency virus 1
agggatcagatatccactgacctttggatggtgc

SEQ ID NO: 37 - DNA, 34 bp, Human immunodeficiency virus 1; identical to SEQ ID NO: 36

SEQ ID NO: 38 - DNA, 34 bp, Human immunodeficiency virus 1
aaaggatagatgtaaaagacaccaaggaagcctt

SEQ ID NO: 39 - DNA, 34 bp, Human immunodeficiency virus 1; identical to SEQ ID NO: 38

SEQ ID NO: 40 - DNA, 1400 bp, Human immunodeficiency virus 1
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagac

SEQ ID NO: 41 - DNA, 1400 bp, Human immunodeficiency virus 1; identical to SEQ ID NO: 40

SEQ ID NO: 42 - DNA, 26 bp, Human immunodeficiency virus 1
gatctgtggatctaccacacacaagg

SEQ ID NO: 43 - DNA, 29 bp, Human immunodeficiency virus 1
ggggtcagatatccactgacctttggatg

SEQ ID NO: 44 - DNA, 29 bp, Human immunodeficiency virus 1
catccggagtacttcaagaactgctgaca

SEQ ID NO: 45 - DNA, 26 bp, Human immunodeficiency virus 1
gccctcagatgctacatataagcagc

SEQ ID NO: 46 - DNA, 26 bp, Human immunodeficiency virus 1
gatctgtaaatctaccacacacaagg

SEQ ID NO: 47 - DNA, 20 bp, Human immunodeficiency virus 1
ggggtcagatatccactgac

SEQ ID NO: 48 - DNA, 13 bp, Human immunodeficiency virus 1
agaactgctgaca

SEQ ID NO: 49 - DNA, 26 bp, Human immunodeficiency virus 1
gccctcagatgctacatataagcagc

SEQ ID NO: 50 - DNA, 26 bp, Human immunodeficiency virus 1
gatctgtggatctaccacacacaagg

SEQ ID NO: 51 - DNA, 24 bp, Human immunodeficiency virus 1
ggggtcagatatccactgacttcc

SEQ ID NO: 52 - DNA, 20 bp, Human immunodeficiency virus 1
tacttcaagaactgctgaca

SEQ ID NO: 53 - DNA, 26 bp, Human immunodeficiency virus 1
gccctcagatgctacatataagcagc

SEQ ID NO: 54 - DNA, 26 bp, Human immunodeficiency virus 1
gatctgtggatctaccacacacaagg

SEQ ID NO: 55 - DNA, 20 bp, Human immunodeficiency virus 1
ggggtcagatatccactgac

SEQ ID NO: 56 - DNA, 20 bp, Human immunodeficiency virus 1
tacttcaagaactgctgaca

SEQ ID NO: 57 - DNA, 26 bp, Human immunodeficiency virus 1
gccctcagatgctacatataagcagc

SEQ ID NO: 58 - DNA, 25 bp, Human immunodeficiency virus 1
atctgtggatctaccacacacaagg

SEQ ID NO: 59 - DNA, 20 bp, Human immunodeficiency virus 1
ggggtcagatatccactgac

SEQ ID NO: 60 - DNA, 20 bp, Human immunodeficiency virus 1
tacttcaagaactgctgaca

SEQ ID NO: 61 - DNA, 26 bp, Human immunodeficiency virus 1
gccctcagatgctacatataagcagc

SEQ ID NO: 62 - DNA, 26 bp, Human immunodeficiency virus 1
gatctgtggatctaccacacacaagg

SEQ ID NO: 63 - DNA, 18 bp, Human immunodeficiency virus 1
ggggtcagatatccactg

SEQ ID NO: 64 - DNA, 10 bp, Human immunodeficiency virus 1
actgctgaca

SEQ ID NO: 65 - DNA, 26 bp, Human immunodeficiency virus 1
gccctcagatgctacatataagcagc

SEQ ID NO: 66 - DNA, 26 bp, Human immunodeficiency virus 1
gatctgtggatctaccacacacaagg

SEQ ID NO: 67 - DNA, 26 bp, Human immunodeficiency virus 1
ggggtcagatatccactgacgtcctc

SEQ ID NO: 68 - DNA, 20 bp, Human immunodeficiency virus 1
tacttcaagaactgctgaca

SEQ ID NO: 69 - DNA, 26 bp, Human immunodeficiency virus 1
gccctcagatgctacatataagcagc

SEQ ID NO: 70 - DNA, 26 bp, Human immunodeficiency virus 1
gatctgtggatctaccacacacaagg

SEQ ID NO: 71 - DNA, 20 bp, Human immunodeficiency virus 1
ggggtcagatatccactgac

SEQ ID NO: 72 - DNA, 20 bp, Human immunodeficiency virus 1
tacttcaagaactgctgaca

SEQ ID NO: 73 - DNA, 26 bp, Human immunodeficiency virus 1
gccctcagatgctacatataagcagc

SEQ ID NO: 74 - DNA, 26 bp, Human immunodeficiency virus 1
gatctgtggatctaccacacacaagg

SEQ ID NO: 75 - DNA, 26 bp, Human immunodeficiency virus 1
ggggtcagatatccactgacgtcctc

SEQ ID NO: 76 - DNA, 20 bp, Human immunodeficiency virus 1
tacttcaagaactgctgaca

SEQ ID NO: 77 - DNA, 26 bp, Human immunodeficiency virus 1
gccctcagatgctacatataagcagc

SEQ ID NO: 78 - DNA, 26 bp, Human immunodeficiency virus 1
gatctgtggatctaccacacacaagg

SEQ ID NO: 79 - DNA, 20 bp, Human immunodeficiency virus 1
ggggtcagatatccactgac

SEQ ID NO: 80 - DNA, 20 bp, Human immunodeficiency virus 1
tacttcaagaactgctgaca

SEQ ID NO: 81 - DNA, 26 bp, Human immunodeficiency virus 1
gccctcagatgctacatataagcagc

SEQ ID NO: 82 - DNA, 26 bp, Human immunodeficiency virus 1
gatctgtggatctaccacacacaagg

SEQ ID NO: 83 - DNA, 24 bp, Human immunodeficiency virus 1
ggggtcagatatccactgacttcc

SEQ ID NO: 84 - DNA, 20 bp, Human immunodeficiency virus 1
tacttcaagaactgctgaca

SEQ ID NO: 85 - DNA, 26 bp, Human immunodeficiency virus 1
gccctcagatgctacatataagcagc